(19) United States
(12) Patent Application Publication
(10) Pub. No.: US 2014/0026255 A1
(43) Pub. Date: Jan. 23, 2014

(54) Transgenic Expression of Archaea Superoxide Reductase

(71) Applicant: NORTH CAROLINA STATE UNIVERSITY, RALEIGH, NC (US)

(72) Inventors: AMY MICHELE GRUNDEN, HOLLY SPRINGS, NC (US); HEIKE INGE ADA SEDEROFF, RALEIGH, NC (US); ROOPA D. YALAMANCHILI, CARY, NC (US)

(21) Appl. No.: 13/925,340

(22) Filed: Jun. 24, 2013

Related U.S. Application Data
(60) Provisional application No. 61/673,564, filed on Jul. 19, 2012.

(51) Int. Cl. C12N 15/82; C12N 15/81; C12N 15/74

(52) U.S. Cl. CPC C12N 15/8273; C12N 15/74; C12N 15/81
USPC 800/278; 424/195.16; 424/755; 424/780; 435/468; 435/471; 435/419; 435/252.3; 435/254.2; 536/23.2; 800/298; 44/605; 426/615; 426/635; 426/629; 426/630; 426/590; 426/598

(57) ABSTRACT

This invention relates to compositions and methods for reducing reactive oxygen species in plants, yeast, algae or bacteria by transforming a plant, yeast or bacteria with a heterologous polynucleotide encoding a superoxide reductase from an archaeon species. The invention also provides methods for protecting a photosynthetic reaction center, for reducing photorespiration and/or for increasing the photosynthetic efficiency of plants or cyanobacteria as well as methods for increasing tolerance to abiotic stress in plants, yeast or bacteria by transforming a plant, yeast, or bacteria with a heterologous polynucleotide encoding a archaeon superoxide reductase. Methods for delaying senescence, reducing lignin polymerization and increasing accessibility of cell wall cellulose to an enzyme in a plant by transforming the plant with a heterologous polynucleotide encoding an archaeon superoxide reductase are also provided. Additionally, transformed plants, yeast and bacteria are provided as well as products produced from the transformed plants, yeast and bacteria.

### STATEMENT OF PRIORITY

This application claims the benefit, under 35 U.S.C. §119 (e), of U.S. Provisional Application No. 61/673,546 was filed on Jul. 19, 2012, the entire contents of which is incorporated by reference herein.

### STATEMENT OF GOVERNMENT SUPPORT

This invention was supported in part by funding provided under Grant No 2009-35318-05024 from the United States Department of Agriculture (USDA), and Grant No DE-AR0000207 from the United States Department of Energy (DOE). The United States government has certain rights in this invention.

### STATEMENT REGARDING ELECTRONIC FILING OF A SEQUENCE LISTING

A Sequence Listing in ASCII text format, submitted under 37 C.F.R. §1.821, entitled 5051-813TS_ST25.txt, 48,434 bytes in size, generated Jun. 19, 2013 and filed via EFS-Web, is provided in lieu of a paper copy. This Sequence Listing is hereby incorporated herein by reference into the specification for its disclosures.

### FIELD OF THE INVENTION

The present invention relates to compositions and methods for reducing reactive oxygen species in a plant, yeast, algae and/or bacteria by transforming the plant, yeast, algae and/or bacteria with a heterologous polynucleotide encoding a superoxide reductase from an archaeon species.

### BACKGROUND

Reactive oxygen species (ROS) are chemically reactive molecules formed due to incomplete reduction of oxygen and include superoxide anions (O²⁻), hydrogen peroxide (H₂O₂), and hydroxyl radicals (HO). ROS are highly reactive due to the presence of unpaired electrons. ROS are natural byproducts of normal metabolism of oxygen in many organisms and play an important role in cell signaling and homeostasis. However, elevated levels of ROS can have detrimental results. The levels of ROS can increase dramatically when an organism is exposed to various environmental stresses such as exposure to heat, excessive light, drought, anoxia, toxins, pathogens, and the like, resulting in oxidative damage and cell death. In plants, for example, oxidative damage from excess ROS can result in reduced photosynthetic efficiency. Plants and other organisms have endogenous ROS metabolizing enzymes such as superoxide dismutase, catalase and peroxidase for preventing the buildup of ROS. However, these endogenous protective mechanisms can be insufficient when the organism experiences environmental stress conditions.

### SUMMARY OF THE INVENTION

In one aspect, the present invention provides a stably transformed plant, plant cell, plant part, yeast cell or bacterial cell comprising a heterologous polynucleotide encoding a superoxide reductase from an archaeon species, wherein said superoxide reductase is localized in the chloroplast, cytosolic membrane, peroxisome, cell wall, and/or mitochondria of said transformed plant, plant cell, plant part, yeast cell or bacterial cell.

In another aspect of the invention, a method of reducing reactive oxygen species in a plant, plant cell, plant part, yeast cell or bacterial cell is provided, the method comprising: introducing into said plant, plant cell, plant part, yeast cell or bacterial cell a heterologous polynucleotide encoding a superoxide reductase (SOR) from an archaeon species to produce a stably transformed plant, plant cell, plant part, yeast cell or bacterial cell, wherein said superoxide reductase is expressed and localized to the chloroplast, cytosolic membrane, peroxisome, cell wall, mitochondria, periplasm and/or as a membrane associated protein of said stably transformed plant, plant cell, plant part, yeast cell or bacterial cell, thereby reducing reactive oxygen species in said stably transformed plant, plant cell, plant part, yeast cell or bacterial cell.

In a further aspect, the present invention provides a method of protecting photosynthetic reaction centers of a plant, plant cell, plant part, or cyanobacteria cell, comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplasts of said stably transformed plant, plant cell, plant part, or cyanobacteria cell, thereby protecting the photosynthetic reaction center of said stably transformed plant, plant cell, plant part, or cyanobacteria cell.

In some aspects of the invention, a method of reducing photorespiration in a plant, plant cell, plant part, or cyanobacteria cell is provided, the method comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplast, the peroxisome, and/or mitochondria of said stably transformed plant, plant cell, plant part, or cyanobacteria cell, thereby reducing photorespiration in said stably transformed plant, plant cell, plant part, or cyanobacteria cell.

In still other aspects of the invention, a method of increasing photosynthetic efficiency in a plant, plant cell, plant part, or cyanobacteria cell is provided, the method comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplasts of said stably transformed plant, plant cell, plant part, or cyanobacteria cell, thereby increasing photosynthetic efficiency in said stably transformed plant, plant cell, plant part, or cyanobacteria cell.

In additional aspects, the present invention provides a method of increasing tolerance to abiotic stress (e.g., heat, high light, drought, ozone, heavy metals, pesticides, herbicides, toxins, and/or anoxia) in a plant, plant cell, plant part, yeast cell or bacterial cell, comprising: introducing into said plant, plant cell, plant part, yeast cell or bacterial cell a heterologous polynucleotide encoding a superoxide reductase from an archaeon species to produce a stably transformed plant, plant cell, plant part, yeast cell or bacterial cell, wherein said superoxide reductase is expressed and localized to the chloroplast, cell wall, mitochondria, periplasm and/or as a membrane associated protein of said stably transformed plant, plant cell, plant part, yeast cell or bacterial cell, thereby increasing tolerance to heat, high light, drought, ozone, heavy metals, pesticides, herbicides, toxins, and/or anoxia in said stably transformed plant, plant cell, plant part, yeast cell or bacterial cell.

In other aspects, the present invention provides a method of reducing lignin polymerization in a plant, plant part and/or plant cell, comprising: introducing into said plant, plant part and/or plant cell a heterologous polynucleotide encoding a superoxide reductase from an archaeon species to produce a stably transformed plant, plant part and/or plant cell, wherein said superoxide reductase is expressed and localized to the cell wall of said stably transformed plant, plant part and/or plant cell, thereby reducing lignin polymerization in said stably transformed plant, plant part and/or plant cell.

A still further aspect of the invention provides a method of increasing accessibility of cell wall cellulose in a plant, plant part and/or plant cell to at least one enzyme, comprising: introducing into said plant, plant part and/or plant cell a heterologous polynucleotide encoding a superoxide reductase from an archaeon species to produce a stably transformed plant, plant part and/or plant cell, wherein said superoxide reductase is expressed and localized to the cell wall of said stably transformed plant, plant part and/or plant cell, thereby increasing accessibility of the cell wall cellulose to at least one enzyme in said stably transformed plant, plant part and/or plant cell.

In an additional aspect, the present invention provides a method of delaying senescence in a plant, plant part, plant cell, bacterium and/or yeast comprising: introducing into said plant, plant part, plant cell, bacterium and/or yeast a heterologous polynucleotide encoding a superoxide reductase from an archaeon species to produce a stably transformed plant, plant part, plant cell, bacterium and/or yeast, wherein said superoxide reductase is expressed and localized to the chloroplast, mitochondria, peroxisome, cytosolic membrane (e.g., cytosolic surface of the plasmamembrane and other endogenous membranes including the nuclear envelope and endoplasmic reticulum (ER)), periplasm and/or as a membrane associated protein of said stably transformed plant, plant part, plant cell, bacterium and/or yeast, thereby delaying the senescence of the stably transformed plant, plant part, plant cell, bacterium and/or yeast.

An additional aspect of the invention provides an isolated polypeptide comprising an amino acid sequence of SEQ ID NO:4. In a further aspect, the invention provides a nucleotide sequence comprising, essentially consisting of, consisting of (a) a nucleotide sequence of SEQ ID NO:2; (b) a nucleotide sequence that encodes a polypeptide comprising an amino acid sequence of SEQ ID NO:4; and/or (c) a nucleotide sequence that differs from the nucleotide sequences of (a) or (b) above due to the degeneracy of the genetic code.

In other aspects, the present invention provides crops produced from the stably transformed plants of the invention as well as products produced from the transformed plants, plant cells, plant parts, yeast cells and/or bacterial cells of this invention.

The foregoing and other objects and aspects of the present invention are explained in detail in the drawings and specification set forth below.

### BRIEF DESCRIPTION OF THE DRAWINGS

FIG. 1 shows vector maps of constructs for plant transformation: pEG100: CTP-SOR, pEG100:CTP-EGFP-SOR and pEG100:CTP-SOR-EGFP.

FIG. 2 shows vector maps of constructs for yeast transformation: pFL36:SOR and pFL36: EGFP-SOR

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Archaea are single celled microorganisms many of which have developed the ability to survive in extreme environments such as high heat and salt (i.e., extremophiles). Pyrococcus furiosus is an extremophilic (hyperthermophilic) species of archaea with optimum growth at 100° C. It is found in hydrothermal vents and is a strict anaerobe at growth permissible temperatures. P. furiosus uses an enzyme, superoxide reductase (SOR), to deal with ROS. P. furiosus SOR has a functional temperature range of about 4° C. to about 100° C. Unlike superoxide dismutase (SOD), which is an endogenous enzyme found in plants, including algae, and in yeast and aerobic bacteria, SOR is more efficient in removing ROS and does so without producing oxygen (thereby reducing the potential for further ROS generation).

Accordingly, the present invention is directed to transgenic plants, plant cells, plant parts, yeast cells and/or bacterial cells comprising a heterologous polynucleotide encoding a superoxide reductase from an archaeon species and methods of reducing reactive oxygen species, protecting photosynthetic reaction centers, reducing photorespiration, increasing photosynthetic efficiency, increasing tolerance to abiotic stress, reducing lignin polymerization and increasing accessibility of cell wall cellulose to at least one enzyme in said transgenic plants, plant cells, plant parts, yeast cells and/or bacterial cells.

Thus, a first aspect of the present invention provides a stably transformed plant, plant cell, plant part, yeast cell or bacterial cell comprising a heterologous polynucleotide encoding a superoxide reductase (SOR) from an archaeon species, wherein said SOR is localized in the chloroplast, cytosol and/or cytosolic membrane, cell wall, peroxisome, mitochondria, periplasm and/or as a membrane associated protein of said transformed plant, plant cell, plant part, yeast cell or bacterial cell. In some embodiments, the heterologous polynucleotide encoding the archaeon SOR is not localized in the cytosol or cytosolic membrane of a plant, plant part or plant cell. In further embodiments, the heterologous polynucleotide encoding the archaeon SOR is not localized in the cytosol and/or cytosolic membrane of a plant, plant part or plant cell when said plant, plant part or plant cell is from Arabidopsis thaliana and/or a higher plant.

In another aspect of the invention, a method of reducing reactive oxygen species in a plant, plant cell, plant part, yeast cell or bacterial cell is provided, the method comprising: introducing into said plant, plant cell, plant part, yeast cell or bacterial cell a heterologous polynucleotide encoding a SOR from an archaeon species to produce a stably transformed plant, plant cell, plant part, yeast cell or bacterial cell, wherein said SOR is expressed and localized to the chloroplast, cytosolic membrane, cell wall, peroxisome, mitochondria, periplasm and/or as a membrane associated protein of said stably transformed plant, plant cell, plant part, yeast cell or bacterial cell, thereby reducing reactive oxygen species in said stably transformed plant, plant cell, plant part, yeast cell or bacterial cell as compared to a control plant, plant cell, plant part, yeast cell or bacterial cell that has not been stably transformed with said heterologous polynucleotide encoding a SOR from an archaeon species. In some embodiments, the heterologous polynucleotide encoding the archaeon SOR is not localized in the cytosol and/or cytosolic membrane of a plant, plant part or plant cell. In further embodiments, the heterologous polynucleotide encoding the archaeon SOR is not localized in the cytosol and/or cytosolic membrane of a plant, plant part or plant cell when said plant, plant part or plant cell is from Arabidopsis thaliana and/or a higher plant.

Methods for detecting and quantifying ROS or oxidized cell components are well known in the art and include, but are not limited to: the nitroblue tetrazolium assay (Fryer et al. J Exp Bot 53: 1249-1254 (2002); Fryer et al. Plant J 33: 691-705 (2003)) and acridan lumigen PS-3 assay (Uy et al. Journal of Biomolecular Techniques 22:95-107 (2011) for detection of superoxide; the ferrous ammonium sulfate/xylenol orange (FOX) method (Wolff, Methods Enzymol 233: 182-189 (1994); Im et al. Plant Physiol 151:893-904 (2009)) for detection of peroxide; the thiobarbituric acid assay (TBA) (Draper and Hadley, Methods Enzymol 186:421-431 (1990); Hodges et al. Planta 207: 604-611 (1999)) and the mass spectrometric determination of peroxidated lipids (Deighton et al. Free Radic Res 27: 255-265 (1997)) for detection of lipid peroxidation; the assay for 8-hydroxy-2′-deoxygunanosine in DNA (Bialkowski and Olinski, Acta Biochim Pol 46: 43-49 (1999)) for the detection of nucleic acid oxidation; and the reaction of oxidized protein with 2,4-dinitrophenylhydrazine (DPNH) (Levine et al. Methods Enzymol 233:346-357 (1994)) for detection of protein oxidation.

In a further aspect, the present invention provides a method of protecting the photosynthetic apparatus and/or surrounding membrane lipids of a plant, plant cell, plant part, or cyanobacteria cell, comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplasts of said stably transformed plant, plant cell, plant part, or the cytosol of a cyanobacteria cell, thereby protecting the photosynthetic apparatus and/or surrounding membrane lipids of said stably transformed plant, plant cell, plant part, or cyanobacteria cell.

A “photosynthetic apparatus and surrounding membrane lipids” is a complex of specific proteins, pigments, lipids and other co-factors that includes the two photosystems and the proteins involved in electron and proton transfer between them as well as the ATPase that function in the primary energy conversion reactions of photosynthesis. During the process of photosynthesis electron transfer reactions are promoted along a series of protein-bound co-factors and it is these electron transfer steps that are the initial phase of a series of energy conversion reactions, ultimately resulting in the production of chemical energy during photosynthesis. Notably, reactive oxygen species can be generated during photosynthetic electron transfer resulting in oxidative damage to the photosynthetic reaction centers. Thus, the present invention protects the photosynthetic apparatus and surrounding membrane lipids by reducing the reactive oxygen species generated during photosynthetic electron transfer.

Methods for measuring “photosynthetic efficiency” or “photosynthesis rate” and thus measuring the protection of photosynthetic apparatus and/or its surrounding membrane lipids are known in the art and include, for example, fluorescence and gas exchange (CO₂, O₂, H₂O) measurements (e.g. Licor), analyzing the chlorophyll content and composition using light spectroscopy, and comparing protein content and turnover of photocenters (Chow et al. Photosynthesis Research: 1-12 (2012) and Hideg et al. Plant and Cell Physiology 49: 1879-1886 (2008)).

In some aspects of the invention, a method of reducing photorespiration in a plant, plant cell, plant part, or cyanobacteria cell is provided, the method comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplast, peroxisome, and/or mitochondria of said stably transformed plant, plant cell, plant part, or cyanobacteria cell, thereby reducing photorespiration in said stably transformed plant, plant cell, plant part, or cyanobacteria cell as compared to a control plant, plant cell, plant part, or cyanobacteria cell that has not been stably transformed with said heterologous polynucleotide encoding a SOR from an archaeon species.

Methods for measuring photorespiration are known in the art. Thus, photorespiration can be indirectly measured by changes in the CO₂-saturation curve using fluorescence and gas exchange measurements (e.g., LiCOR) or via ¹⁸O₂ incorporation. Alternatively, determining the ratio of serine to glycine in actively photosynthesizing leaves can be used to measure photorespiration. Other ways that changes in photorespiration can be shown include comparing biomass productivity or photosynthesis under different CO₂:O₂ environments. See, e.g., Hideg et al. Plant and Cell Physiology 49: 1879-1886 (2008); and Berry et al. Plant Physiol 62:954-967 (1978).

In still other aspects of the invention, a method of increasing photosynthetic efficiency in a plant, plant cell, plant part, or cyanobacteria cell is provided, the method comprising: introducing into said plant, plant cell, plant part, or cyanobacteria cell a heterologous polynucleotide encoding a superoxide reductase from an archaeon species to produce a stably transformed plant, plant cell, plant part, or cyanobacteria cell, wherein said superoxide reductase is expressed and localized to the chloroplasts of said stably transformed plant, plant cell, plant part, or cyanobacteria cell, thereby increasing photosynthetic efficiency in said stably transformed plant, plant cell, plant part, or cyanobacteria cell as compared to a control plant, plant cell, plant part, or cyanobacteria cell that has not been stably transformed with said heterologous polynucleotide encoding a SOR from an archaeon species.

Photosynthetic efficiency is the fraction of light energy converted into chemical energy during photosynthesis. Saturating pulse fluorescence measurements can be used to measure photosynthetic efficiency. CO₂ and O₂ exchange methods can also be used. A number of plant and algae studies have been done, which demonstrate that photosynthetic efficiency decreases when plants are exposed to ROS (Ganesh et al. Biotechnol Bioeng 96(6):1191-8 (2007); Zhang and Xing. Plant Cell Physiology 49(7):1092-1111 (2008).

Reactive oxygen species (ROS) are generated in the cells of aerobic organisms during normal metabolic processes and have been identified to have an important role in cell signaling and homeostasis. However, high levels of ROS can be detrimental to an organism's cell structure and metabolism often resulting in cell death (i.e., oxidative stress). Most organisms have endogenous mechanisms for protecting them from potential damage by ROS, including enzymes such as superoxide dismutase, catalase and peroxide, and small antioxidant molecules. However, under conditions of abiotic stress, the levels of ROS can rise significantly making the endogenous protective mechanisms insufficient. By stably introducing a heterologous polynucleotide encoding SOR from an archaeon species into the cells of plants, bacteria and yeast as described herein, said plants, yeasts and bacteria stably expressing the SOR have increased tolerance to the environmental stresses that induce ROS production.

Thus, in additional aspects, the present invention provides a method of increasing tolerance to abiotic stress (e.g., heat, high light, drought, ozone, heavy metals, pesticides, herbicides, toxins, and/or anoxia) in a plant, plant cell, plant part, yeast cell or bacterial cell, comprising: introducing into said plant, plant cell, plant part, yeast cell or bacterial cell a heterologous polynucleotide encoding a superoxide reductase from an archaeon species to produce a stably transformed plant, plant cell, plant part, yeast cell or bacterial cell, wherein said superoxide reductase is expressed and localized to the chloroplast, cell wall, mitochondria, periplasm and/or as a membrane associated protein, of said stably transformed plant, plant cell, plant part, yeast cell or bacterial cell, thereby increasing tolerance to heat, high light, drought, ozone, heavy metals, pesticides, herbicides and/or anoxia in said stably transformed plant, plant cell, plant part, yeast cell or bacterial cell as compared to a control plant, plant cell, plant part, yeast cell or bacterial cell that has not been stably transformed with said heterologous polynucleotide encoding a SOR from an archaeon species.

“Abiotic stress” or “environmental stress” as used herein means any outside, nonliving, physical or chemical factors or conditions that induce ROS production. Thus, in some embodiments of the invention, an abiotic or environmental stress can include, but is not limited to, high heat, high light, drought, ozone, heavy metals, pesticides, herbicides, toxins, and/or anoxia (i.e., root flooding).

In other embodiments, environmental/abiotic stress for organisms used in fermentation can include but is not limited to, high metabolic flux and/or high fermentation product accumulation. In other embodiments, environmental/abiotic stress for organisms used in biofuel production can include, but is not limited to, biofuel product accumulation. Thus, in some embodiments, a method for increasing tolerance to high metabolic flux, high fermentation product accumulation and/or biofuel product accumulation in a plant cell, yeast cell or bacterial cell is provided, the method comprising introducing into said plant cell, yeast cell or bacterial cell a heterologous polynucleotide encoding a superoxide reductase from an archaeon species to produce a stably transformed plant cell, yeast cell or bacterial cell, wherein said superoxide reductase is expressed and localized to the chloroplast, the cell wall, mitochondria, periplasm and/or as a membrane associated protein of said stably transformed plant cell, yeast cell or bacterial cell, thereby increasing tolerance to high metabolic flux, high fermentation product accumulation and/or biofuel product accumulation in said stably transformed plant cell, yeast cell or bacterial cell as compared to a control plant cell, yeast cell or bacterial cell that has not been transformed with said heterologous polynucleotide encoding a superoxide reductase from an archaeon species.

Parameters for the abiotic stress factors are species specific and even variety specific and therefore vary widely according to the species/variety exposed to the abiotic stress. Thus, for example, while one species may be severely impacted by a high temperature of 23° C., another species may not be impacted until at least 30° C., and the like. Temperatures above 30° C. result in, for example, dramatic reductions in the yields of many plant crops including algae. This is due to reductions in photosynthesis that begin at approximately 20-25° C., and the increased carbohydrate demands of crops growing at higher temperatures. The critical temperatures are not absolute, but vary depending upon such factors as the acclimatization of the organism to prevailing environmental conditions. In addition, because organisms are often exposed to multiple abiotic stresses at one time, the interaction between the stresses affects the response. For example, damage to a plant from excess light occurs at lower light intensities as temperatures increase beyond the photosynthetic optimum. Water stressed plants are less able to cool overheated tissues due to reduced transpiration, further exacerbating the impact of excess (high) heat and/or excess (high) light intensity. Thus, the particular parameters for high/low temperature, light intensity, drought and the like, which can negatively impact an organism will vary with species, variety, degree of acclimatization and the exposure to a combination of environmental conditions.

In an additional aspect, the present invention provides a method of delaying senescence in a plant, plant part, plant cell, bacterium and/or yeast, comprising: introducing into said plant, plant part and/or plant cell a heterologous polynucleotide encoding a superoxide reductase from an archaeon species to produce a stably transformed plant, plant part, plant cell, bacterium and/or yeast, wherein said superoxide reductase is expressed and localized to the chloroplast, mitochondria, peroxisome, cytosolic membrane (e.g., cytosolic surface of the plasma-membrane and other endogenous membranes including the nuclear envelope and endoplasmic reticulum (ER)), periplasm and/or as a membrane associated protein of said stably transformed plant, plant part, plant cell, bacterium and/or yeast, thereby delaying the senescence of the stably transformed plant, plant part, plant cell, bacterium and/or yeast as compared to a plant, plant part, plant cell, bacterium and/or yeast that has not been transformed with said heterologous polynucleotide encoding a superoxide reductase from an archaeon species.

In other aspects, the present invention provides a method of reducing lignin polymerization in a plant, plant part and/or plant cell, comprising: introducing into said plant, plant part and/or plant cell a heterologous polynucleotide encoding a superoxide reductase from an archaeon species to produce a stably transformed plant, plant part and/or plant cell, wherein said superoxide reductase is expressed and localized to the cell wall of said stably transformed plant, plant part and/or plant cell, thereby reducing lignin polymerization in said stably transformed plant, plant part and/or plant cell as compared to a control plant, plant cell and/or plant part that has not been stably transformed with said heterologous polynucleotide encoding a SOR from an archaeon species.

Methods for measuring reduced lignin polymerization are known in the art. Such methods include, but are not limited to, histochemical staining (Nakano et al. The Detection of Lignin Methods in Lignin Chemistry. Berlin: Springer-Verlag (1992)). Lignin content can also be determined using the Klason procedure (Dence et al. Lignin Determination. Berlin: Springer-Verlag (1992)). In addition, NMR (Kim et al. Bio. Res. 1:56-66 (2008)) or thioacidolysis procedure (Lapierre et al. Res. Chem. Intermed. 21:397-412 (1995)) followed by GC-MS or LC-MS can be used for quantification of lignin monomers.

Lignin polymerization occurs through the radical coupling of hydroxycinnamyl subunits (i.e., monolignols, e.g., coniferyl (CA), sinapyl (SA), and p-coumaryl alcohols (p-CA)). Monolignols require ROS for polymerization (Boerjan et al. Annu. Rev. Plant Biol. 54:519-546 (2003)). Lignin polymers are deposited predominantly in the walls of secondarily thickened cells, making them rigid and impervious. Further, the presence of the lignin polymers in the cell wall reduces the accessibility of the cell wall polysaccharides (cellulose and hemicellulose) to microbes and microbial degradation. As a consequence of its ability to protect the cellulose and hemicellulose in the cell wall from microbial degradation, the presence of lignin is also a limiting factor in the process of converting of plant biomass to biofuels. However, in representative embodiments, the present invention provides methods of reducing lignin polymerization by stably introducing into the cell wall of a plant or plant part, a heterologous polynucleotide encoding a SOR from an archaeon species, thereby reducing the ROS and reducing lignin polymerization in said plant, plant part and/or plant cell. Further, a reduction in lignin polymerization in a plant, plant part and/or plant cell provides the enzymes used in biofuel production greater accessibility to the cellulose and hemicellulose.

Thus, a further aspect of the invention provides a method of increasing accessibility of cell wall cellulose in a plant, plant part and/or plant cell to at least one enzyme (e.g., 1, 2, 3, 4, 5, 6, 7, 8, 9, 10, etc), comprising: introducing into said plant, plant part and/or plant cell a heterologous polynucleotide encoding a superoxide reductase from an archaeon species to produce a stably transformed plant, plant part and/or plant cell, wherein said superoxide reductase is expressed and localized to the cell wall of said stably transformed plant, plant part and/or plant cell, thereby increasing accessibility of the cell wall cellulose to at least one enzyme in said stably transformed plant, plant part and/or plant cell as compared to a control plant, plant part and/or plant cell that has not been stably transformed with said heterologous polynucleotide encoding a SOR from an archaeon species. In some representative embodiments, the increased accessibility can be for at least one cell wall degrading enzyme including, but not limited to, a cellulase and/or a hemicellulase. Cellulose accessibility and digestibility can be determined by directly probing cellulase binding and activity using a fluorescently-tagged cellobiohydrolase (See, for example, Jeoh et al. Biotechnol Bioeng. 98:112-122 (2007))

The terms “increase,” “increasing,” “increased,” “enhance,” “enhanced,” “enhancing,” and “enhancement” (and grammatical variations thereof), as used herein, describe an elevation in the photosynthetic efficiency of a plant, plant cell, plant part, or cyanobacteria cell, an elevation in the tolerance to abiotic (e.g., environmental) stress in a plant, plant cell, plant part, yeast cell or bacterial cell, and/or an elevation in the accessibility of cell wall cellulose in a plant and/or plant part to at least one enzyme as a result of the introduction of a heterologous polynucleotide encoding a SOR from an archaeon species into the plant cell, plant, plant part, yeast cell or bacterial cell. This increase can be observed by comparing the increase in the organism transformed with the heterologous polynucleotide encoding said SOR to the appropriate control (e.g., the same organism lacking (i.e., not transformed with) the heterologous polynucleotide encoding said SOR from an archaeon species). Thus, as used herein, the terms “increase,” “increasing,” “increased,” “enhance,” “enhanced,” “enhancing,” and “enhancement” (and grammatical variations thereof), and similar terms indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control (e.g., a plant, plant part, plant cell, yeast cell, bacterial cell that does not comprise said heterologous polynucleotide encoding SOR from an archaeon species).

As used herein, the terms “reduce,” “reduced,” “reducing,” “reduction,” “diminish,” “suppress,” and “decrease” (and grammatical variations thereof), describe, for example, a decrease in the reactive oxygen species in a plant, plant cell; plant part, yeast cell or bacterial cell, a decrease in photorespiration in a plant, plant cell, plant part, or cyanobacteria cell and/or a decrease in lignin polymerization in a plant, plant part, and/or plant cell as compared to a control as described herein. Thus, as used herein, the terms “reduce,” “reduces,” “reduced,” “reduction,” “diminish,” “suppress,” and “decrease” and similar terms mean a decrease of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, and the like, or any range therein, as compared to a control (e.g., a plant, plant part, plant cell, yeast cell and/or bacterial cell that does not comprise a heterologous polynucleotide encoding SOR from an archeaon species).

As used herein, the terms “express,” “expresses,” “expressed” or “expression,” and the like, with respect to a nucleotide sequence (e.g., RNA or DNA) indicates that the nucleotide sequence is transcribed and, optionally, translated. Thus, a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers, and the like.

In some embodiments, the archaeon species can be a species from the genus Pyrococcus, a species from the genus Thermococcus, or a species from the genus Archaeoglobus. In other embodiments, the archaeon species can be Pyrococcus fitriosus and the heterologous polynucleotide encoding a SOR can be a nucleotide sequence of SEQ ID NO:1 or SEQ ID NO:2. In some embodiments, the heterologous polynucleotide encoding a SOR encodes an amino acid sequence of SEQ ID NO:3 or SEQ ID NO:4. Thus, in some embodiments, the invention provides a nucleotide sequence comprising, essentially consisting of, consisting of (a) a nucleotide sequence of SEQ ID NO:2; (b) a nucleotide sequence that encodes a polypeptide comprising an amino acid sequence of SEQ ID NO:4; and/or (c) a nucleotide sequence that differs from the nucleotide sequences of (a) or (b) above due to the degeneracy of the genetic code. In other embodiments, the invention provides an isolated polypeptide comprising, essentially consisting of or consisting of an amino acid sequence of SEQ ID NO:4.

In some embodiments, the heterologous polynucleotide encoding a SOR is operably associated with a targeting nucleotide sequence encoding a signal peptide that targets the heterologous SOR to the cytosol, cytosolic membrane (e.g., cytosolic surface of the plasma-membrane and other endogenous membranes including the nuclear envelope and endoplasmic reticulum), chloroplast, cell wall, peroxisome, mitochondria, periplasm and/or as a membrane associated protein. The signal sequence may be operably linked at the N- or C-terminus of the nucleic acid molecule. In some embodiments, the heterologous polynucleotide encoding a SOR is not operably associated with a targeting nucleotide sequence that encodes a signal peptide targeting said SOR to the cytosol and/or cytosolic membrane. In other embodiments, the heterologous polynucleotide encoding a SOR is not operably associated with a targeting nucleotide sequence that encodes a signal peptide targeting said SOR to the cytosolic membrane. In some particular embodiments, when the targeting nucleotide sequence encodes a signal peptide that targets the SOR to the cytosol and/or cytosolic membrane, the plant, plant part and/or plant cell is not from a higher plant. In other embodiments, when the targeting nucleotide sequence encodes a signal peptide that targets the SOR to the cytosol and/or cytosolic membrane, the plant, plant part and/or plant cell is not Arabidopsis thaliana or not from Arabidopsis thaliana.

Signal peptides (and the targeting nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC Bioinformatics 6:249 (2005)(available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inrafr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (www.cbs.dtu.dk/services/TargetP/); predicts the subcellular location of eukaryotic proteins—the location assignment is based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971 (2007); Zuegge et al. 280(1-2):19-26 (2001); Neuberger et al. J Mol Biol. 328(3):567-79 (2003); and Neuberger et al. J Mol Biol. 328(3):581-92 (2003)).

Exemplary signal peptides include, but are not limited to those provided in Table 1.

TABLE 1
Amino acid sequences of representative signal peptides.

| Source | Sequence | Target |
|---|---|---|
| Rubisco small subunit | MASSVLSSAAVATRSNVAQANMVAPFTGLKSAASFPVSR | chloroplast |
| (tobacco) | KQNLDITSIASNGGRVQC (SEQ ID NO: 5) | |
| Saccharomyces | MLSLRQSIRFFKPATRTLCSSRYLL (SEQ ID NO: 7) | mitochondria |
| cerevisiae cox4 | | |
| Arabidopsis aconitase | MYLTASSSASSSIIRAASSRSSSLFSFRSVLSPSVSSTSPSSLL | mitochondria |
| | ARRSFGTISPAFRRWSHSFHSKPSPFRFTSQIRA (SEQ ID | |
| | NO: 9) | |
| Yeast aconitase | MLSARSAIKRPIVRGLATV (SEQ ID NO: 40) | mitochondria |
| Arabidopsis proline-rich | MRILPKSGGGALCLLFVFALCSVAHS (SEQ ID NO: 11) | cell wall/secretory |
| protein 2 (AT2G21140) | | pathway |
| PTS-2 (conserved in | RLX₅HL (SEQ ID NO: 13) | peroxisome |
| eukaryotes) | MRLSIHAEHL (SEQ ID NO: 14) | |
| | SKL | |
| Arabidopsis presequence | MLRTVSCLASRSSSSLFFRFFRQFPRSYMSLTSSTAALRYPSRNLR | mitochondria and |
| protease1 (AT3G19170) | RISSPSVAGRRLLLRRGLRIPSAAVRSYNGQFSRLSVRA | chloroplast |
| | (SEQ ID NO: 16) | |
| Chlamydomonas | MALVARPVLSARVAASRPRVAARKAVRVSAKYGEN (SEQ ID | chloroplast |
| reinhardtii-(Stroma- | NO: 41) | |
| targeting cTPs: | MQALSSRVNIAAKPQRAQRLVVRAEEVKA (SEQ ID NO: 42) | |
| photosystem I (PSI) | MQTLASRPSLRASARVAPRRAPRVAVVTKAALDPQ (SEQ ID | |
| subunits P28, P30, P35 | NO: 43) | |
| and P37, respectively) | MQALATRPSAIRPTKAARRSSVVVRADGFIG (SEQ ID NO: 44) | |
| C. reinhardtii- | MAFALASRKALQVTCKATGKKTAAKAAAPKSSGVEFYGPNRAK | chloroplast |
| chlorophyll a/b protein | WLGPYSEN (SEQ ID NO: 45) | |
| (cabII-1) | | |
| C. reinhardtii-Rubisco | MAAVIAKSSVSAAVARPARSSVRPMAALKPAVKAAPVAAPAQA | chloroplast |
| small subunit | NQMMVWT (SEQ ID NO: 46) | |
| C. reinhardtii-ATPase-γ | MAAMLASKQGAFMGRSSFAPAPKGVASRGSLQVVAGLKEV | chloroplast |
| | (SEQ ID NO: 47) | |
| Escherichia coli phoA | MKQSTIAKAKKPLLFTPVTKA (SEQ ID NO: 48) | periplasm |
| alkaline phosphatase | | |
| Arabidopsis thaliana | CVVQ (SEQ ID NO: 34) | membrane |
| abscisic acid receptor | | |
| PYL 10 | | |

X₅ means any five amino acids can be present in the sequence to target the protein to the peroxisome (e.g. RLAVAVAHL, SEQ ID NO: 49).

Thus, in representative embodiments of the invention, a heterologous polynucleotide encoding a SOR to be expressed in a plant, plant cell, plant part can be operably linked to a chloroplast targeting sequence encoding a chloroplast signal peptide, optionally wherein said chloroplast signal peptide is encoded by an amino acid sequence that includes, but is not limited to, the amino acid sequence of SEQ ID NO:5, SEQ ID NO:41, SEQ ID NO:42, SEQ ID NO:43, SEQ ID NO:44, SEQ ID NO:45, SEQ ID NO:46, or SEQ ID NO:47.

In other embodiments of the invention, a heterologous polynucleotide encoding a SOR to be expressed in a plant, plant part, plant cell or yeast cell can be operably linked to a mitochondrial targeting sequence encoding a mitochondrial signal peptide, optionally wherein said mitochondrial signal peptide is encoded by an amino acid sequence that includes, but is not limited to, the amino acid sequence of SEQ ID NO:7, SEQ ID NO:9, or SEQ ID NO:40.

In further embodiments, a heterologous polynucleotide encoding a SOR to be expressed in a plant, plant part, plant cell, yeast cell, or bacterial cell can be operably linked to a cell wall targeting sequence encoding a cell wall signal peptide, optionally wherein said cell wall signal peptide is encoded by an amino acid sequence that includes, but is not limited to, the amino acid sequence of SEQ ID NO:11.

In still further embodiments of the invention, a heterologous polynucleotide encoding a SOR to be expressed in a plant, plant part, plant cell, or a yeast cell can be operably linked to a peroxisomal targeting sequence encoding a peroxisomal signal peptide, optionally wherein said peroxisomal signal peptide is encoded by an amino acid sequence that includes, but is not limited to, the amino acid sequence of SEQ ID NO:13, SEQ ID NO:14, or SKL.

In additional embodiments, a heterologous polynucleotide encoding a SOR to be expressed in a bacterial cell can be operably linked to a periplasmic targeting sequence encoding a periplasmic signal peptide, optionally wherein said periplasmic signal peptide is encoded by an amino acid sequence that includes, but is not limited to, the amino acid sequence of SEQ ID NO:48.

In some embodiments, a heterologous polynucleotide encoding a SOR to be expressed in a plant, plant part, plant cell, yeast cell or bacterial cell can be operably linked to a membrane targeting sequence encoding a membrane signal peptide, optionally wherein said membrane signal peptide is encoded by an amino acid sequence that includes, but is not limited to, the amino acid sequence of SEQ ID NO:34. In some embodiments, wherein when the heterologous polynucleotide encoding a SOR is targeted to a membrane, the SOR can be either linked directly to the membrane or to the membrane via a linkage to a membrane associated protein. In representative embodiments, a membrane associated protein includes but is not limited to the plasma membrane NADH oxidase (RbohA) (for respiratory burst oxidase homolog A) (Keller et al. The Plant Cell Online 10: 255-266 (1998)), annexin1 (ANN1) from Arabidopsis thaliana (Laohavisit et al. Plant Cell Online 24: 1522-1533 (2012)), and/or the nitrate transporter CHL1 (AtNRT1.1) (Tray et al. “The Role of Plasma Membrane Nitrogen Transporters in Nitrogen Acquisition and Utilization,” In, The Plant Plasma Membrane 19:223-236 Springer Berlin/Heidelberg (2011)).

Targeting to a membrane is similar to targeting to an organelle. Thus, specific sequences on a protein (targeting sequences or motifs) can be recognized by a transporter, which then imports the protein into an organelle or in the case of membrane association, the transporter can guide the protein to and associate it with a membrane. Thus, for example, a specific cysteine residue on a C-terminal motif of a protein can be modified posttranslation where an enzyme (prenyltransferases) then attaches a hydrophobic molecule (geranylgeranyl or farnesyl) (See, e.g., Running et al. Proc Natl Acad Sci USA 101: 7815-7820 (2004); Maurer-Stroh et al. Genome Biology 4:212 (2003)). This hydrophobic addition guides and associates the protein to a membrane (in case of the cytosol, the membrane would be the plasmamembrane or the cytosolic site of the nuclear membrane (Polychronidou et al. Molecular Biology of the Cell 21: 3409-3420 (2010)). More specifically, in representative embodiments, a protein prenyltransferase can catalyze the covalent attachment of a 15-carbon farnesyl or 20-carbon geranylgeranyl isoprenoid to C-terminal cysteines of selected proteins carrying a CaaX motif where C=cysteine; a=aliphatic amino acid; x=any amino acid. For plants, this motif most often is CVVQ. The addition of prenyl groups facilitates membrane association and protein-protein interactions of the prenylated proteins.

In still other embodiments of the invention, a signal peptide can direct a SOR to more than one organelle (e.g., dual targeting). Thus, in some embodiments, a signal peptide that can target the heterologous SOR to more than one organelle is encoded by an amino acid sequence that includes, but is not limited to, the amino acid sequence of SEQ ID NO:16.

In some embodiments, the heterologous polynucleotide encoding an SOR from an archaeon species can be comprised within an expression cassette. As used herein, “expression cassette” means a recombinant nucleic acid molecule comprising at least one nucleotide sequence of interest (e.g., the heterologous polynucleotide encoding SOR), wherein said heterologous polynucleotide is operably associated with at least a control sequence (e.g., a promoter). Thus, some embodiments of the invention provide expression cassettes designed to express a polynucleotide encoding an archaeon SOR. In this manner, for example, a promoter operably associated with a heterologous polynucleotide encoding an SOR from an archaeon species (e.g., SEQ ID NO:1 or SEQ ID NO:2), and/or functional fragment thereof) are provided in expression cassettes for expression in a plant, plant part, plant cell, bacterial cell and/or yeast cell.

An expression cassette comprising a heterologous polynucleotide encoding an SOR may be chimeric, meaning that at least one of its components is heterologous with respect to at least one of its other components. An expression cassette may also be one that is naturally occurring but has been obtained in a recombinant form useful for heterologous expression.

Any promoter useful for initiation of transcription in a cell of a plant, yeast or bacteria can be used in the expression cassettes of the present invention. A “promoter,” as used herein, is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” A promoter can be identified in and isolated from the organism to be transformed and then inserted into the nucleic acid construct to be used in transformation of the organism.

The choice of promoter will vary depending on the temporal and spatial requirements for expression, and also depending on the host cell to be transformed. Thus, for example, expression of the heterologous polynucleotide encoding an archaeon SOR can be in any plant, plant part, (e.g., in leaves, in stalks or stems, in ears, in inflorescences (e.g. spikes, panicles, cobs, etc.), in roots, seeds and/or seedlings, and the like), plant cells (including algae cells), yeast cells, or bacterial cells. For example, in the case of a multicellular organism such as a plant where expression in a specific tissue or organ is desired, a tissue-specific or tissue preferred promoter can be used (e.g., a root specific/preferred promoter). In contrast, where expression in response to a stimulus is desired a promoter inducible by stimuli or chemicals can be used. Where continuous expression at a relatively constant level is desired throughout the cells or tissues of an organism a constitutive promoter can be chosen.

Thus, promoters useful with the invention include, but are not limited to, those that drive expression of a nucleotide sequence constitutively, those that drive expression when induced, and those that drive expression in a tissue- or developmentally-specific manner. These various types of promoters are known in the art. Promoters can be identified in and isolated from the plant, yeast, or bacteria to be transformed and then inserted into the expression cassette to be used in transformation of the plant, yeast, or bacteria.

Non-limiting examples of a promoter include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol. Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol. Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci. USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Particularly useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters useful with plants are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant 111:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In some particular embodiments, promoters useful with algae include, but are not limited to, the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)), the promoter of the σ⁷⁰-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (See, e.g., De Cosa et al. Nat. Biotechnol. 19:71-74 (2001); Daniell et al. BMC Biotechnol. 9:33 (2009); Muto et al. BMC Biotechnol. 9:26 (2009); Surzycki et al. Biologicals 37:133-138 (2009)).

In some embodiments, promoters useful with bacteria and yeast include, but are not limited to, a constitutive promoter (e.g., lpp (lipoprotein gene)) and/or an oxidative stress inducible promoter (e.g., a superoxide dismutase or a catalase promoter).

Thus, in some embodiments, a promoter useful with yeast can include, but is not limited to, a promoter from phosphoglycerate kinase (PGK), glyceraldehyde-3-phosphate dehydrogenase (GAP), triose phosphate isomerase (TP1), galactose-regulon (GAL1, GAL10), alcohol dehydrogenase (ADH1, ADH2), phosphatase (PHO5), copper-activated metallothionine (CUP1), MFα1, PGK/α2 operator, TPI/α2 operator, GAP/GAL, PGK/GAL, GAP/ADH2, GAP/PHO5, iso-1-cytochrome c/glucocorticoid response element (CYC/GRE), phosphoglycerate kinase/angrogen response element (PGK/ARE), transcription elongation factor EF-1α (TEF1), triose phosphate dehydrogenase (TDH3), phosphoglycerate kinase 1 (PGK1), pyruvate kinase 1 (PYK1), and/or hexose transporter (HXT7) (See, Romanos et al. Yeast 8:423-488 (1992); and Partow et al. Yeast 27:955-964 (2010)).

In additional embodiments, a promoter useful with bacteria can include, but is not limited to, L-arabinose inducible (araBAD, P_{BAD}) promoter, any lac promoter, L-rhamnose inducible (rhaP_{BAD}) promoter, T7 RNA polymerase promoter, trc promoter, tac promoter, lambda phage promoter (p_{L},p_{L}-9G-50), anhydrotetracycline-inducible (tetA) promoter, up, lpp, phoA, recA, proU, cst-1, cadA, nar, lpp-lac, cspA, T7-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, α-amylase (Pamy), Ptms, P43 (comprised of two overlapping RNA polymerase a factor recognition sites, σA, σB), Ptms, P43, rplK-rplA, ferredoxin promoter, and/or xylose promoter. (See, K. Terpe Appl. Microbiol, Biotechnol. 72:211-222 (2006); Hannig et al. Trends in Biotechnology 16:54-60 (1998); and Srivastava Protein Expr Purif 40:221-229 (2005)).

In addition to promoters operably linked to a heterologous polynucleotide of the present invention (e.g., polynucleotide encoding an archaeal SOR (e.g., a P. furiosus SOR, SEQ ID NO:1, SEQ ID NO:2)) an expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences, translation termination sequences, and polyadenylation signal sequences, as described herein.

Thus, in some embodiments of the present invention, the expression cassettes can include at least one intron. An intron useful with this invention can be an intron identified in and isolated from a plant, yeast, or bacteria to be transformed and then inserted into the expression cassette to be used in transformation of the plant, yeast, or bacteria. As would be understood by those of skill in the art, the introns as used herein comprise the sequences required for self excision and are incorporated into the nucleic acid constructs in frame. An intron can be used either as a spacer to separate multiple protein-coding sequences in one nucleic acid construct, or an intron can be used inside one protein-coding sequence to stabilize the mRNA. If they are used within a protein-coding sequence, they are inserted “in-frame” with the excision sites included.

Non-limiting examples of introns useful with the present invention can be introns from the RuBisCO small subunit (rbcS) gene, the RuBisCO large subunit (rbcL) gene, the actin gene, the nitrate reductase gene (nr), the duplicated carbonic anhydrase gene 1 (Tdca1), the psbA gene, the atpA gene, or any combination thereof.

In some embodiments of the invention, an expression cassette can comprise an enhancer sequence. Enhancer sequences can be derived from, for example, any intron from any highly expressed gene. In particular embodiments, an enhancer sequence usable with this invention includes, but is not limited to, the nucleotide sequence of ggagg (e.g., ribosome binding site).

An expression cassette also can optionally include a transcriptional and/or translational termination region (i.e., termination region) that is functional in plants, yeast or bacteria. A variety of transcriptional terminators are available for use in expression cassettes and are responsible for the termination of transcription beyond the heterologous polynucleotide of interest and correct mRNA polyadenylation. The termination region may be native to the transcriptional initiation region, may be native to the operably linked nucleotide sequence of interest, may be native to the host cell, or may be derived from another source (i.e., foreign or heterologous to the promoter, the nucleotide sequence of interest, the host cell, or any combination thereof). Non-limiting examples of transcriptional terminators useful for plants can be a CAMV 35S terminator, a tm1 terminator, a nopaline synthase terminator and/or a pea rbcs E9 terminator, a RubisCo small subunit gene 1 (TrbcS1) terminator, an actin gene (Tactin) terminator, a nitrate reductase gene (Tnr) terminator, and/or aa duplicated carbonic anhydrase gene 1 (Tdca1) terminator.

Further non-limiting examples of terminators useful with this invention for expression of SOR or other heterologous polynucleotides in algae include a terminator of the psbA gene (TpsbA), a terminator of the psaA gene (encoding an apoprotein of photosystem I) (TpsaA), a terminator of the psbD gene (TpsbD), a RuBisCo large subunit terminator (TrbcL), a terminator of the σ⁷⁰-type plastid rRNA gene (Trrn), and/or a terminator of the ATPase alpha subunit gene (TatpA).

Non-limiting examples of terminators for use with bacteria can be from trp, hom-trpB, lysA, thrB, and/or sodA.

An expression cassette of the invention also can include a nucleotide sequence for a selectable marker, which can be used to select a transformed plant, plant part, plant cell, yeast cell or bacteria cell. As used herein, “selectable marker” means a nucleotide sequence that when expressed imparts a distinct phenotype to a plant, plant part, plant cell, yeast cell or bacterial cell expressing the marker and thus allows such a transformed plant, plant part, plant cell, yeast cell or bacterial cell to be distinguished from that which does not have the marker. Such a nucleotide sequence may encode either a selectable or screenable marker, depending on whether the marker confers a trait that can be selected for by chemical means, such as by using a selective agent (e.g., an antibiotic, herbicide, or the like), or whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding aadA (i.e., spectinomycin and streptomycin resistance), a nucleotide sequence encoding neo (i.e., kanamycin resistance), a nucleotide sequence encoding aphA6 (i.e., kanamycin resistance), a nucleotide sequence encoding nptII (i.e., kanamycin resistance), a nucleotide sequence encoding bar (i.e., phosphinothricin resistance), a nucleotide sequence encoding cat (i.e., chloramphenicol resistance), a nucleotide sequence encoding badh (i.e., betaine aldehyde resistance), a nucleotide sequence encoding egfp, (i.e., enhanced green fluorescence protein), a nucleotide sequence encoding gfp (i.e., green fluorescent protein), a nucleotide sequence encoding luc (i.e., luciferase), a nucleotide sequence encoding ble (bleomycin resistance), a nucleotide sequence encoding ereA (erythromycin resistance), and any combination thereof.

Further examples of selectable markers useful with the invention include, but are not limited to, a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding Bla that confers ampicillin resistance; or a nucleotide sequence encoding aequorin which may be employed in calcium-sensitive bioluminescence detection (Prasher et al. (1985) Biochem. Biophys. Res. Comm. 126:1259-1268), and/or any combination thereof. One of skill in the art is capable of choosing a suitable selectable marker for use in an expression cassette of this invention.

An expression cassette comprising a heterologous polynucleotide encoding an SOR also can include polynucleotides that encode other desired traits. Such desired traits can be polynucleotides which confer high light tolerance, increased drought tolerance, increased flooding tolerance, increased tolerance to soil contaminants, increased CO2 uptake, increased CO2 assimilation, modification of carbon flux, increased yield, modified fatty acid composition of the lipids, increased oil production in seed, increased and modified starch production in seeds, increased and modified protein production in seeds, modified tolerance to herbicides and pesticides, production of terpenes, increased seed number, increased biomass of the roots, increased and/or modified biomass of the stem (trees), increased and/or modified biomass of the leaves, reduced photorespiration, and/or other desirable traits for agriculture or biotechnology.

In particular embodiments, in addition to a heterologous polynucleotide encoding an SOR, an expression cassette of this invention can further comprise an archaeal rubrerythrin reductase for conversion of hydrogen peroxide to water. Rubrerythrin reductase is an iron-dependent peroxidase that functions in vivo to remove the peroxide produced by superoxide reductase. Thus, a further embodiment of the invention includes a stably transformed plant comprising an expression cassette that comprises a SOR and a rubrerythrin reductase. In some embodiments, the SOR and rubrerythrin reductase are co-localized (i.e., they are expressed and targeted to the same or similar position in the transformed cell).

In some embodiments, an archaeal rubrerythrin reductase can be from Pyrococcus furiosus. In further embodiments, an archaeal rubrerythrin reductase can be encoded by the nucleotide sequence of:

(SEQ ID NO: 50)
atggtcgtgaaaagaacaatgactaaaaagttcttggaagaagcctttgcaggcgaaagcatggcccatatgaggtatttgatctttgcc
gagaaagctgaacaagaaggatttccaaacatagccaagctgttcagggcaatagcttacgcagagtttgttcacgctaaaaaccactt
catagctctaggaaaattaggcaaaactccagaaaacttacagatgggaatagagggagaaacgttcgaagttgaggaaatgtaccc
agtatacaacaaagccgcagaattccaaggagaaaaggaagcagttagaacaacccactatgattagaggcggagaagatccacg
ctgaactctatagaaaggcaaaagagaaagctgagaaaggggaagacattgaaataaagaaagtttacatatgcccaatctgtggata
caccgctgttgatgaggctccagaatactgtccagtttgtggagctccaaaagaaaagttcgttgtctttgaatga

In still further embodiments, an archaeal rubrerythrin reductase can comprise, consist essentially of, or consist of the amino acid sequence of:

(SEQ ID NO: 511)
MVVKRTMTKKFLEEAFAGESMAHMRYLIFAEKAEQEGFPNIAKLFRAIA
YAEFVHAKNHFIALGKLGKTPENLQMGIEGETFEVEEMYPVYNKAAEFQ
GEKEAVRTTHYALEAEKIHAELYRKAKEKAEKGEDIEIKKVYICPICGY
TAVDEAPEYCPVCGAPKEKFVVFE.

In still other embodiments, an expression cassette can further encode green fluorescent protein (GFP).

Such polynucleotides can be stacked with any combination of nucleotide sequences to create plants, plant parts, plant cells, yeast cells or bacterial cells having the desired phenotype. Stacked combinations can be created by any method including, but not limited to, any conventional methodology (e.g., cross breeding for plants), or by genetic transformation. If stacked by genetic transformation, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

By “operably linked” or “operably associated,” it is meant that the indicated elements are functionally related to each other, and are also generally physically related. Thus, the term “operably linked” or “operably associated” as used herein, refers to nucleotide sequences on a single nucleic acid molecule that are functionally associated. Therefore, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Any plant (or groupings of plants, for example, into a genus or higher order classification) can be employed in practicing the present invention including an angiosperm, a gymnosperm, a monocot, a dicot, a C3, C4, CAM plant, a microalgae, and/or a macroalgae.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell.

In some embodiments of this invention, a plant, plant part or plant cell can be from a genus including, but not limited to, the genus of Camelina, Sorghum, Gossypium, Brassica, Allium, Armoracia, Poa, Agrostis, Lolium, Festuca, Calamogrostis, Deschampsia, Spinacia, Beta, Pisum, Chenopodium, Helianthus, Pastinaca, Daucus, Petroselium, Populus, Prunus, Castanea, Eucalyptus, Acer, Quercus, Salix, Juglans, Picea, Pinus, Abies, Lemna, Wolffia, Spirodela, Oryza or Gossypium.

In other embodiments, a plant, plant part or plant cell can be from a species including, but not limited to, the species of Camelina alyssum (Mill.) Thell., Camelina macrocarpa Andrz. ex DC., Camelina rumelica Velen., Camelina sativa (L.) Crantz, Sorghum bicolor (e.g., Sorghum bicolor L. Moench), Gossypium hirsutum, Brassica oleracea, Brassica rapa, Brassica napus, Raphanus sativus, Armoracia rusticana, Allium sative, Allium cepa, Populus grandidentata, Populus tremula, Populus tremuloides, Prunus serotina, Prunus pensylvanica, Castanea dentate, Populus balsamifer, Populus deltoids, Acer Saccharum, Acer nigrum, Acer negundo, Acer rubrum, Acer saccharinum, Acer pseudoplatanus or Oryza sativa. In additional embodiments, the plant, plant part or plant cell can be, but is not limited to, a plant of, or a plant part, or plant cell from wheat, barley, oats, turfgrass (bluegrass, bentgrass, ryegrass, fescue), feather reed grass, tufted hair grass, spinach, beets, chard, quinoa, sugar beets, lettuce, sunflower (Helianthus annuus), peas (Pisum sativum), parsnips (Pastinaca sativa), carrots (Daucus carota), parsley (Petroselinum crispum), duckweed, pine, spruce, fir, eucalyptus, oak, walnut, or willow. In particular embodiments, the plant, plant part and/or plant cell can be from Camelina sativa. In other particular embodiments, the plant, plant part and/or plant cell is not from Arabidopsis thaliana.

In further embodiments, a plant and/or plant cell can be an algae or algae cell from a class including, but not limited to, the class of Bacillariophyceae (diatoms), Haptophyceae, Phaeophyceae (brown algae), Rhodophyceae (red algae) or Glaucophyceae (red algae). In still other embodiments, a plant and/or plant cell can be an algae or algae cell from a genus including, but not limited to, the genus of Achnanthidium, Actinella, Nitzschia, Nupela, Geissleria, Gomphonema, Planothidium, Halamphora, Psammothidium, Navicula, Eunotia, Stauroneis, Chlamydomonas, Dunaliella, Nannochloris, Nannochloropsis, Scenedesmus, Chlorella, Cyclotella, Amphora, Thalassiosira, Phaeodactylum, Chrysochromulina, Prymnesium, Thalassiosira, Phaeodactylum, Glaucocystis, Cyanophora, Galdieria, or Porphyridium. Additional nonlimiting examples of genera and species of diatoms useful with this invention are provided by the US Geological Survey/Institute of Arctic and Alpine Research at westerndiatoms.colorado.edu/species.

Any bacterium can be employed in practicing the present invention. In particular embodiments, a bacterial cell can be from a phylum that includes, but not limited to, the phylum of Cyanobacteria or can be from a genus including, but not limited to, the genus of Bacillus, Lactobacillus, Lactococcus, Streptococcus, Pseudomonas, Corynebacterium, Escherichia or Clostridium. In some embodiments, a bacterial cell can be Escherichia coli.

Further, any yeast in which heterologous expression of a SOR is useful can be used with the methods of this invention. In some representative embodiments, a yeast cell can be from a genus including, but not limited to, the genus of Saccharomyces, Saccharomycodes, Kluyveromyces, Pichia, Candida, Zygosaccharomyces or Hanseniaspora. In other embodiments, a yeast cell can be from a species including, but not limited to, the species of Saccharomyces cerevisiae, S. uvarum (carlsbergensis), S. diastaticus, Saccharomycodes ludwigii, Kluyveromyces marxianus, Pichia pastoris, Candida stellata, C. pulcherrima, Zygosaccharomyces fermentati, or Hanseniaspora uvarum.

Any nucleotide sequence to be transformed into a plant, plant part, plant cell, yeast cell or bacterial cell can be modified for codon usage bias using species specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications for the nucleotide sequences for selection are determined by comparing the species specific codon usage table with the codons present in the native nucleotide sequences. In those embodiments in which each of codons in native nucleotide sequence for selection are sufficiently used, then no modifications are needed (e.g., a frequency of more than 30% for a codon used for a specific amino acid in that species would indicate no need for modification). In other embodiments, wherein up to 3 nucleotides have to be modified in the nucleotide sequence, site-directed mutagenesis can be used according to methods known in the art (Zheng et al. Nucleic Acids Res. 32:e115 (2004); Dammai, Meth. Mol. Biol. 634:111-126 (2010); Davis and Vierstra. Plant Mol. Biol. 36(4): 521-528 (1998)). In still other embodiments, wherein more than three nucleotide changes are necessary, a synthetic nucleotide sequence can be generated using the same codon usage as the highly expressed genes that were used to develop the codon usage table.

The term “transformation” as used herein refers to the introduction of a heterologous polynucleotide into a cell. Transformation of a plant, plant part, plant cell, yeast cell and/or bacterial cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols that are well known in the art.

A heterologous polynucleotide encoding a SOR from an archaeon species as described herein and/or fragments thereof, and/or any combination thereof, can be introduced into the cell of a plant, yeast or bacteria by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants, yeast and bacteria are well known and routine in the art and are described throughout the literature. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)). General guides to the transformation of yeast include Guthrie and Fink (1991) (Guide to yeast genetics and molecular biology. In Methods in Enzymology, (Academic Press, San Diego) 194:1-932) and guides to methods related to the transformation of bacteria include Aune and Aachmann (Appl. Microbiol. Biotechnol 85:1301-1313 (2010)).

A polynucleotide therefore can be introduced into a plant, plant part, plant cell, yeast cell and/or bacterial cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant, bacteria or yeast, as part of a breeding protocol.

In some embodiments, when a plant part or plant cell is stably transformed, it can then be used to regenerate a stably transformed plant comprising the heterologous polynucleotide encoding an SOR from an archaeon species in its genome. Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

The particular conditions for transformation, selection and regeneration of a plant can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention further provides a product produced from the stably transformed plant, plant cell, plant part, yeast cell or bacterial cell of the invention. In some embodiments, the product produced can include but is not limited to biofuel, food, drink, animal feed, fiber, and/or pharmaceuticals.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

As used herein, the terms “fragment” when used in reference to a polynucleotide will be understood to mean a nucleic acid molecule or polynucleotide of reduced length relative to a reference nucleic acid molecule or polynucleotide and comprising, consisting essentially of and/or consisting of a nucleotide sequence of contiguous nucleotides identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment according to the invention may be, where appropriate, included in a larger polynucleotide of which it is a constituent.

As used herein, a “functional” polypeptide or “functional fragment” is one that substantially retains at least one biological activity normally associated with that polypeptide. In particular embodiments, the “functional” polypeptide or “functional fragment” substantially retains all of the activities possessed by the unmodified peptide. By “substantially retains” biological activity, it is meant that the polypeptide retains at least about 50%, 55%, 60%, 65%, 70%, 75%, 80%, 85%, 90%, 95%, 96%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Thus, for example, a functional fragment of an archaeon SOR polypeptide is a polypeptide that retains at least 50% or more SOR activity.

An “isolated” nucleic acid molecule or nucleotide sequence or nucleic acid construct or double stranded RNA molecule of the present invention is generally free of nucleotide sequences that flank the nucleic acid of interest in the genomic DNA of the organism from which the nucleic acid was derived (such as coding sequences present at the 5′ or 3′ ends). However, the nucleic acid molecule of this invention can include some additional bases or moieties that do not deleteriously or materially affect the basic structural and/or functional characteristics of the nucleic acid molecule.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose. In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely or fully complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules either along the full length of the molecules or along a portion or region of the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least at about 50%, 60%, 70%, 80% or 90% of their nucleotides. In some embodiments, the two nucleic acid sequences can be complementary at least at about 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid molecule or nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous polynucleotide includes a nucleotide sequence derived from and inserted into the same natural, original cell type, but which is present in a non-natural state, e.g. present in a different copy number, and/or under the control of different regulatory sequences than that found in the native state of the nucleic acid molecule.

As used herein, the terms “transformed” and “transgenic” refer to any plant, plant part, plant cell, yeast cell or bacterial cell that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transgene” as used herein, refers to any nucleotide sequence used in the transformation of an organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic yeast, or transgenic bacterium, is an organism into which a transgene has been delivered or introduced and the transgene can be expressed in the transgenic organism to produce a product, the presence of which can impart an effect and/or a phenotype in the organism.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleotide sequences have at least 70%, 75%, 80%, 85%, 90% or 95% sequence identity. In some embodiments, the two nucleotide sequences can have at least 80%, 85%, 90%, 91%, 92%, 93%, 94%, 95%, 96%, 97%, 98%, 99% or 100% sequence identity. Thus, for example, a homolog of an SOR from an archaeon species of this invention can have at least about 80%, 85%, 90%, 91%, 92%, 93%, 94%, 95%, 96%, 97%, 98%, 99% or 100% sequence identity to said SOR of this invention.

Two nucleotide sequences can also be considered to be substantially identical when the two sequences hybridize to each other under stringent conditions. A nonlimiting example of “stringent” hybridization conditions include conditions represented by a wash stringency of 50% formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol, 215:403-410 (1990)); version 2.0 or higher of BLAST programs allows the introduction of gaps (deletions and insertions) into alignments; for peptide sequence BLASTX can be used to determine sequence identity; and for polynucleotide sequence BLASTN can be used to determine sequence identity.

Accordingly, the present invention further provides nucleotide sequences having substantial sequence identity to the nucleotide sequences of the present invention (e.g., the polynucleotides encoding a SOR from an archaeon species). Substantial sequence similarity or identity means at least 70%, 75%, 80%, 81%, 82%, 83%, 84%, 85%, 86%, 87%, 88%, 89%, 90%, 91%, 92%, 93%, 94%, 95%, 96%, 97%, 98%, 99% and/or 100% similarity or identity with another nucleotide sequence.

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

### Example 1

### Plants

Plants are continually challenged by environmental stresses that result in increased production of reactive oxygen species (ROS, e.g., superoxide and hydrogen peroxide). ROS can induce a switch from primary to secondary metabolism and can ultimately lead to plant tissue death. Like other aerobic organisms, plants have ROS scavenging enzymes, such as superoxide dismutase (SOD), peroxidase and catalases that help prevent the production and buildup of toxic free radicals.

Pyrococcus furiosus is an extremophilic (hyperthermophile) species of archaea with optimum growth at 100° C. It is found in hydrothermal vents and is a strict anaerobe. P. furiosus uses superoxide reductase (SOR—functional range of 4-100° C.) rather than SOD to deal with ROS. Unlike SOD, the endogenous plant enzyme, SOR is more efficient in removing ROS and does so without producing oxygen (i.e. reducing the potential for further ROS generation). Thus, for example, transformation of a plant to express an archaeon SOR in the chloroplast can assist in the reduction of ROS, thereby protecting the transgenic plant's photosynthetic reaction centers, lowering O₂ content, which in turn helps to reduce photorespiration, and reduce expression of defense mechanisms that diminish photosynthetic electron flux.

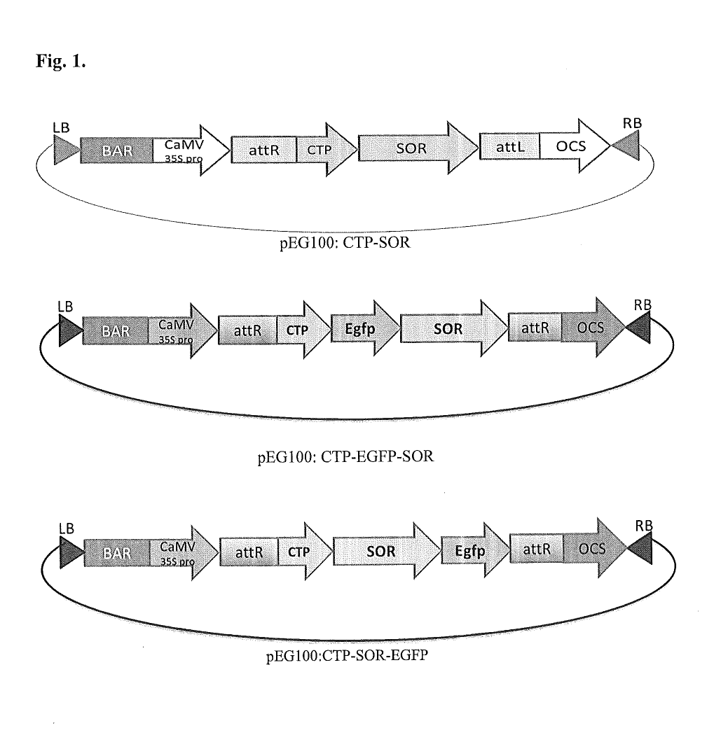

Camelina sativa plants stably transformed with a heterologous polynucleotide encoding a P. furiosus SOR and expressing the SOR in the chloroplasts are assessed for protection of the photosynthetic apparatus and its surrounding membrane lipids from oxidative damage, reduced photosynthetic electron flux, and increased tolerance to abiotic stresses (e.g., drought, heat, high light). Transgenic plants in which SOR is targeted to the chloroplast, mitochondria, peroxisome, and/or cytosolic membrane are assessed for delayed senescence and increased abiotic stress tolerance and biomass production. Transgenic plants expressing P. furiosus SOR in cell walls are assessed for reduction in lignin polymerization and for an increased accessibility of cell wall cellulose to at least one cell wall degrading enzyme such as cellulase and hemicellulase. Exemplary vectors for transformation of plants are provided in FIG. 1.

### Example 2

### Yeast

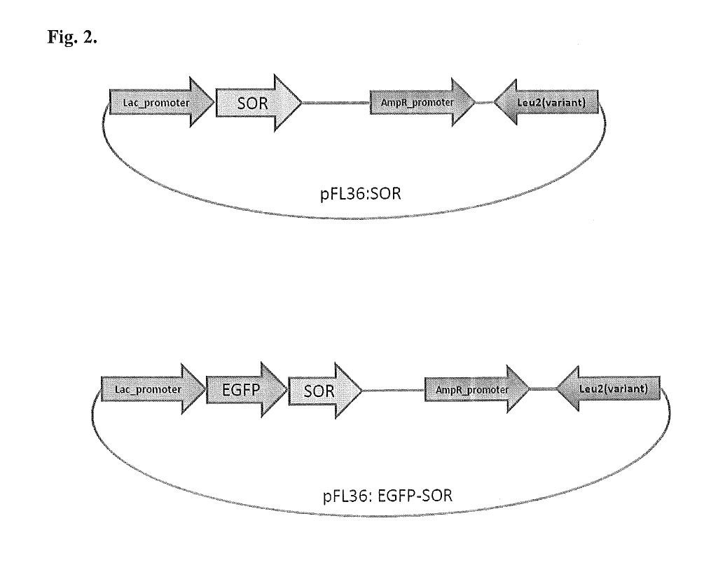

Industrial yeast strains generate reactive oxygen species (ROS, e.g., superoxide and hydrogen peroxide) in response to fermentation product accumulation and metabolic flux. ROS can oxidatively damage cellular components and can ultimately lead to cell death. Like other facultative aerobic organisms, yeast have ROS scavenging enzymes, such as superoxide dismutase (SOD), peroxidase and catalases that help prevent the production and buildup of toxic free radicals. However, transformation of yeast with archaeal SOR (targeted to the mitochondria, cytosol or as a membrane associated protein) would help further protect yeast from the ROS generated by metabolic flux and fermentation product buildup (ex. ethanol). Exemplary vectors for transformation of yeast are provided in FIG. 2.

### Example 3

### Bacteria

Industrial bacterial strains, such as those used for biofuel production (cyanobacteria, E. coli, Clostridium), generate ROS in response to metabolic flux and biofuel molecule accumulation. ROS can irreversibly damage bacterial macromolecules and cell structures and can ultimately lead to bacterial cell death. Transformation of bacteria with archaeal SOR (targeted either to the cytosol, to the periplasm, or as a membrane-associated protein) would aid in protecting the bacterial cells from ROS generated by metabolic flux and biofuel molecule accumulation. In some embodiments, when the SOR to be expressed in a bacterial cell is targeted to the periplasm, the periplasmic targeting protein can be encoded by the nucleotide sequence of atgaaacagagcaccattgcgaaagcgaaaaaaccgctgctgtttaccccggtgaccaaagcg (SEQ ID NO:52) or the amino acid sequence of MKQSTIAKAKKPLLFTPVTKA (SEQ ID NO:48).

### Example 4

### Preparation of P. furiosus Superoxide Reductase Polynucleotide for Plant Transformation

The gene encoding P. furiosus superoxide reductase (SOR) was amplified using polymerase chain reaction (PCR), pfu DNA polymerase and the indicated primers (forward primers; 5′-CAC CAT GAT TAG TGA AAC CAT AAG-3′, SEQ ID NO:53, for cloning into pENTR/D/TOPO and 5′-ATG ATT AGT GAA ACC ATA AG-3′, SEQ ID NO:54, for cloning into pCR8/GW/TOPO, and reverse primer; 5′-TCA CTC TAA AGT GAC TTC GTT TTC-3′, SEQ ID NO:55) to amplify the coding region of SOR. The resulting amplification product was subcloned into pENTR/D/TOPO and pCR8/GW/TOPO entry vectors (Invitrogen, Carlsbad, Calif.) and then into pEG100, pEG103 and pEG104 destination vectors (Functional Genomics Division of the Department of Plant Systems Biology, Gent, Belgium) using LR recombination reactions according to the manufacturer's instructions (Invitrogen). The resulting constructs were as follows:

pEG103:SOR—C terminal GFP
pEG104:SOR—N terminal YFP
pEG100:SOR—no tags
pEG100:EGFP-SOR—N terminal fusion with EGFP

These above constructs enabled production of green fluorescence protein (GFP)-fusion-SOR proteins under the control of a CaMV 35S promoter in plants. Recombinant plasmids were transformed into Agrobacterium tumefaciens GV3101 using electroporation and then transformed into Camelina by vacuum infiltration of the inflorescences (Lu et al. Plant Cell Reports 27:273-278 (2008)). Four independent transformed lines were further selected. Stable expression of the transgene was monitored by RT-PCR and immunoblotting as described below.

SOR nucleotide and amino acid sequences:
(1) Nucleotide sequence of P. furiosus SOR:
(SEQ ID NO: 1)
atgattagtgaaaccataagaagtggggactggaaaggagaaaagcacgtccccgttatagagtatgaaagagaaggggagcttgtt
aaagttaaggtgcaggttggtaaagaaatcccgcatccaaacaccactgagcaccacatcagatacatagagctttatttcttaccagaa
ggtgagaactttgtttaccaggttggaagagttgagtttacagctcacggagagtctgtaaacggcccaaacacgagtgatgtgtacac
agaacccatagcttactttgtgctcaagactaagaagaagggcaagctctatgctcttagctactgtaacatccacggcctttgggaaaa
cgaagtcactttagagtga
(2) Amino acid sequence of P. furiosus SOR:
(SEQ ID NO: 3)
Met I S E T I R S G D W K G E K H V P V I E Y E R E G E L V K V K V Q V G K E I P H
P N T T E H H I R Y I E L Y F L P E G E N F V Y Q V G R V E F T A H G E S V N G P N
T S D V Y T E P I A Y F V L K T K K K G K L Y A L S Y C N I H G L W E N E V T L E
Stop
(3) Nucleotide sequence of P. furiosus SOR variant:
(SEQ ID NO: 2)
atgattagtgaaaccataagaagtggggactggaaaggagaaaagcacgtccccgttatagagtatgaaagagaaggggagcttgtt
aaagttaaggtgcaggttggtaaagaaatcccgcatccaaacaccactgagcaccacatcagatacatagagattatttcttaccagaa
ggtgagaactttgtttaccaggttggaagagttgagtttacagctcacggagagtctgtaaacggcccaaacacgagtgatgtgtacac
agaacccatagcttactttgtgacaagactaagaagaagggcaagctctatgctcttagcgactgtaacatccacggcctttgggaaaa
cgaagtcactttagagtga
(4) Amino acid sequence of P. furiosus SOR variant:
(SEQ ID NO: 4)
Met I S E T I R S G D W K G E K H V P V I E Y E R E G E L V K V K V Q V G K E I P H
P N T T E H H I R Y I E L Y F L P E G E N F V Y Q V G R V E F T A H G E S V N G P N
T S D V Y T E P I A Y F V L K T K K K G K L Y A L S D C N I H G L W E N E V T L E
Stop
Targeting sequences:
(1) Chloroplast signal sequence:
(SEQ ID NO: 5)
MASSVLSSAAVATRSNVAQANMVAPFTGLKSAASFPVSRKQNLDITSIASNGGRVQC
(SEQ ID NO: 6)
atggcgagcagcgtgctgagcagcgcggcggtggcgacccgcagcaacgtggcgcaggcgaacatggtggcgccg
tttaccggcctgaaaagcgcggcgagctttccggtgagccgcaaacagaacctggatattaccagcattgcgagc
aacggcggccgcgtgcagtgc
(2) Mitochondrial signal sequence
(SEQ ID NO: 7)
MLSLRQSIRFFKPATRTLCSSRYLL
(SEQ ID NO: 8)
atgctgagcctgcgccagagcattcgcttttttaaaccggcgacccgcaccctgtgcagcagccgctatctgctg
(SEQ ID NO: 9)
MYLTASSSASSSIIRAASSRSSSLFSFRSVLSPSVSSTSPSSLLARRSFGTISPAFRRWSHS
FHSKPSPFRFTSQIRA
(SEQ ID NO: 10)
atgtatctgaccgcgagcagcagcgcgagcagcagcattattcgcgcggcgagcagccgcagcagcagcctgtttagctttcgcag
cgtgctgagcccgagcgtgagcagcaccagcccgagcagcctgctggcgcgccgcagctttggcaccattagcccggcgtttcgcc
gctggagccatagctttcatagcaaaccgagcccgtttcgctttaccagccagattcgcgcg
(3) Cell wall signal sequence
(SEQ ID NO: 11)
MRILPKSGGGALCLLFVFALCSVAHS
(SEQ ID NO: 12)
atgcgcattctgccgaaaagcggcggcggcgcgctgtgcctgctgtttgtgtttgcgctgtgcagcgtggcgcat
agc
(4) Peroxisome signal sequence
(SEQ ID NO: 13)
RLXXXXXHL
(SEQ ID NO: 14)
MRLSIHAEHL
(SEQ ID NO: 15)
atgcgcctgagcattcatgcggaacatctg
SKL
agcaaactg
(5) Dual signal sequence for mitochondria and chloroplast
(SEQ ID NO: 16)
MLRTVSCLASRSSSSLFFRFFRQFPRSYMSLTSSTAALRVPSRNLRRISSPSVAGRRL
LLRRGLRIPSAAVRSVNGQFSRLSVRA
(SEQ ID NO: 17)
atgctgcgcaccgtgagctgcctggcgagccgcagcagcagcagcctgttttttcgcttttttcgccagtttccg
cgcagctatatgagcctgaccagcagcaccgcggcgctgcgcgtgccgagccgcaacctgcgccgcattagcagc
ccgagcgtggcgggccgccgcctgctgctgcgccgcggcctgcgcattccgagcgcggcggtgcgcagcgtgaac
ggccagtttagccgcctgagcgtgcgcgcg
Vectors for plant transformation
(1) Exemplary constructs for chloroplast targeting
(A) Chloroplast targeted SOR (pEG100: CTP-SOR)
(SEQ ID NO: 18)
MASSVLSSAAVATRSNVAQANMVAPFTGLKSAASFPVSRKQNLDITSIASNGGRV
QCMISETIRSGDWKGEKHVPVIEYEREGELVKVKVQVGKEIPHPNTTEHHIRYIELYF
LPEGENFVYQVGRVEFTAHGESVNGPNTSDVYTEPIAYFVLKTKKKGKLYALSYCNI
HGLWENEVTLE
(SEQ ID NO: 19)
atggcgagcagcgtgctgagcagcgcggcggtggcgacccgcagcaacgtggcgcaggcgaacatggtggcgccg
tttaccggcctgaaaagcgcggcgagctttccggtgagccgcaaacagaacctggatattaccagcattgcgagc
aacggcggccgcgtgcagtgcatgattagcgaaaccattcgcagcggcgattggaaaggcgaaaaacatgtgccg
gtgattgaatatgaacgcgaaggcgaactggtgaaagtgaaagtgcaggtgggcaaagaaattccgcatccgaac
accaccgaacatcatattcgctatattgaactgtattttctgccggaaggcgaaaactttgtgtatcaggtgggc
cgcgtggaatttaccgcgcatggcgaaagcgtgaacggcccgaacaccagcgatgtgtataccgaaccgattgcg
tattttgtgctgaaaaccaaaaaaaaaggcaaactgtatgcgctgagctattgcaacattcatggcctgtgggaa
aacgaagtgaccctggaa
(B) Chloroplast targeted SOR with EGFP as N terminal fusion
pEG100: CTP-EGFP-SOR nucleotide sequence (chloroplast targeting peptide-enhanced green
fluorescent protein-superoxide reductase (CTP-EGFP-SOR))
(SEQ ID NO: 20)
MASSVLSSAAVATRSNVAQANMVAPFTGLKSAASFPVSRKQNLDITSIASNGGRV
QCMQMVSKGEELFTGVVPILVELDGDVNGHKFSVSGEGEGDATYGKLTLKFICTTG
KLPVPWPTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQKNGIKVN
FKIRHNIEDGSVQLADHYQQNTPIGDGPVLLPDNHYLSTQSALSKDPNEKRDHMVLL
EFVTAAGITLGMDELYKLSSMISETIRSGDWKGEKHVPVIEYEREGELYKYKYQV
GKEIPHPNTTEHHIRYIELYFLPEGENFVYQVGRVEFTAHGESVNGPNTSDVYTE
PIAYFVLKTKKKGKLYALSYCNIHGLWENEVTLE
(SEQ ID NO: 21)
atggcttcctcagttctttcctctgcagcagttgccacccgcagcaatgttgctcaagctaacatggttgcacctttcactggcctt
aagtcagctgcctcattccctgtttcaaggaagcaaaaccttgacatcacttccattgccagcaacggcggaagagtgcaatg
catgcagatggtgagcaagggcgaggagctgttcaccggggtggtgcccatcctggtcgagctggacggcgacgtaaacggccac
aagttcagcgtgtccggcgagggcgagggcgatgccacctacggcaagctgaccctgaagttcatctgcaccaccggcaagctgcc
cgtgccctggcccaccacgtgaccaccctgacctacggcgtgcagtgatcagccgctaccccgaccacatgaagcagcacgactt
cttcaagtccgccatgcccgaaggctacgtccaggagcgcaccatcttatcaaggacgacggcaactacaagacccgcgccgagg
tgaagttcgagggcgacaccctggtgaaccgcatcgagctgaagggcatcgacttcaaggaggacggcaacatcctggggcacaa
gctggagtacaactacaacagccacaacgtctatatcatggccgacaagcagaagaacggcatcaaggtgaacttcaagatccgcca
caacatcgaggacggcagcgtgcagctcgccgaccactaccagcagaacacccccatcggcgacggccccgtgctgctgcccga
caaccactacctgagcacccagtccgccctgagcaaagaccccaacgagaagcgcgatcacatggtcctgctggagttcgtgaccg
ccgccgggatcactctcggcatggacgagctgtacaagatgattagtgaaaccataagaagtggggactggaaaggagaaaagcac
gtccccgttatagagtatgaaagagaaggggagcttgttaaagttaaggtgcaggttggtaaagaaatcccgcatccaaacaccactg
agcaccacatcagatacatagagctttatttcttaccagaaggtgagaactttgtttaccaggttggaagagttgagtttacagctcacgg
agagtctgtaaacggcccaaacacgagtgatgtgtacacagaacccatagcttactagtgctcaagactaagaagaagggcaagctc
tatgctcttagctactgtaacatccacggcctttgggaaaacgaagtcactttagagtga
(C) pEG100: CTP-SOR-EGFP Sequence
(SEQ ID NO: 22)
MASSVLSSAAVATRSNVAQANMVAPFTGLKSAASFPVSRKQNLDITSIASNGGRV
QCMQMISETIRSGDWKGEKHVPVIEYERFGELVKVKVQVGKEIPHPNTTEHHIRYIE
LYFLPEGENFVYQVGRVEFTAHGESVNGPNTSDVYTEPIAYFVLKTKKKGKLYALSY
CNIHGLWENEVTLEMVSKGEELFTGVVPILVELDGDVNGHKFSVSGEGEGDATYGK
LTLKFICTTGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNSNHVYIMAD
KQKNGIKVNFKIRHNIEDGSVQLADHYQQNTPIGDGPVLLPDNHYLSTQSALSKDPN
EKRDHMVLLEFVTAAGITLGMDELYK
(SEQ ID NO: 23)
atggcttcctcagttctttcctctgcagcagttgccacccgcagcaatgttgctcaagctaacatggttgcacctttcactggcctt
aagtcagctgcctcattccctgtttcaaggaagcaaaaccttgacatcacttccattgccagcaacggcggaagagtgcaatg
catgcagatgattagtgaaaccataagaagtggggactggaaaggagaaaagcacgtccccgttatagagtatgaagagaagggg
agcttgttaaagttaaggtgcaggttggtaaagaaatcccgcatccaaacaccactgagcaccacatcagatacatagagctttatttctt
accagaaggtgagaactttgtttaccaggttggaagagttgagtttacagctcacggagagtctgtaaacggcccaaacacgagtgat
gtgtacacagaacccatagcttactttgtgctcaagactaagaagaagggcaagctctatgctcttagctactgtaacatccacggccttt
gggaaaacgaagtcactttagagtgaatggtgagcaagggcgaggagctgttcaccggggtggtgcccatcctggtcgagctggac
ggcgacgtaaacggccacaagttcagcgtgtccggcgagggcgagggcgatgccacctacggcaagctgaccctgaagttcatct
gcaccaccggcaagctgcccgtgccctggcccaccctcgtgaccaccctgacctacggcgtgcagtgcttcagccgctaccccgac
cacatgaagcagcacgacttcttcaagtccgccatgcccgaaggctacgtccaggagcgcaccatcttcttcaaggacgacggcaac
tacaagacccgcgccgaggtgaagttcgagggcgacaccctggtgaaccgcatcgagctgaagggcatcgacttcaaggaggacg
gcaacatcctggggcacaagctggagtacaactacaacagccacaacgtctatatcatggccgacaagcagaagaacggcatcaag
gtgaacttcaagatccgccacaacatcgaggacggcagcgtgcagctcgccgaccactaccagcagaacacccccatcggcgacg
gccccgtgctgctgcccgacaaccactacctgagcacccagtccgccctgagcaaagaccccaacgagaagcgcgatcacatggt
cctgctggagttcgtgaccgccgccgggatcactctcggcatggacgagctgtacaag
(D) Chioroplast targeted SOR with yellow fluorescent protein (YFP) as N terminal
fusion (CTP-YFP-SOR amino acid sequence)
(SEQ ID NO: 24)
MASSVLSSAAVATRSNVAQANMVAPFTGLKSAASFPVSRKQNLDITSIASNGGRV
QCMGKGEELFTGVVPILVELDGDVNGHKFSVSGEGEGDATYGKLTLKFICTTGKLPV
PWPTLVTTFGYGLQCFARYPDHMKQHDFFKSAMPEGYVQERTIFFKDDGNYKTRAE
VKFEGDTLVNRIELKGIDFKEDGNILGHKLEYNYNSHNVYIMADKQKNGIKVNFKIR
HNIEDGSVQLADHYQQNTPIGDGPVLLPDNHYLSYQSALSKDPNEKRDHMVLLEFV
TAAGITLGMDELYKMISETIRSGDWKGEKHVPVIEYEREGELVKVKVQVGKEIP
HPNTTEHHIRYIELYFLPEGENFVYQVGRVEFTAHGESVNGPNTSDVYTEPIAYF
VLKTKKKGKLYALSYCNIHGLWENEVTLE
(SEQ ID NO: 25)
atggcgagcagcgtgctgagcagcgcggcggtggcgacccgcagcaacgtggcgcaggcgaac
atggtggcgccgtttaccggcctgaaaagcgcggcgagctttccggtgagccgcaaacagaacctggatatta
agcattgcgagcaacggcggccgcgtgcagtgcatgggcaaaggcgaagaactgtttaccggcgtggtgccgatt
ctggtggaactggatggcgatgtgaacggccataaatttagcgtgagcggcgaaggcgaaggcgatgccacctat
ggcaaactgaccctgaaatttatttgcaccaccggcaaactgccggtgccgtggccgaccctggtgaccattt
ggctatggcctgcagtgctttgcgcgctatccggatcatatgaaacagcatgatttttttaaaagcgcgatg
gaaggctatgtgcaggaacgcaccattttttttaaagatgatggcaactataaaacccgcgcggaagtgaaattt
gaaggcgataccctggtgaaccgcattgaactgaaaggcattgattttaaagaagatggcaacattctgggccat
aaactggaatataactataacagccataacgtgtatattatggcggataaacagaaaaacggcattaaagtgaac
tttaaaattcgccataacattgaagatggcagcgtgcagctggcggatcattatcagcagaacaccccgattggc
gatggcccggtgctgctgccggataaccattatctgagctatcagagcgcgctgagcaaagatccgaacgaaaaa
cgcgatcatatggtgctgctggaatttgtgaccgcggcgggcattaccctgggcatggatgaactgtataaaat
attagcgaaaccattcgcagcggcgattggaaaggcgaaaaacatgtgccggtgattgaatatgaacgcggc
gaactggtgaaagtgaaagtgcaggtgggcaaagaaattccgcatccgaacaccaccgaacatcatattcgctat
attgaactgtattttctgccggaaggcgaaaactttgtgtatcaggtgggccgcgtggaatttaccgCgCatgc
gaaagcgtgaacggcccgaacaccagcgatgtgtataccgaaccgattgcgtattttgtgctgaaaaccaa
aaaggcaaactgtatgcgctgagctattgcaacattcatggcctgtgggaaaacgaagtgaccctggaa

Maps of exemplary vectors for chloroplast transformation are provided in FIGS. 1A-C (pEG100:CTP-SOR, pEG100:CTP-EGFP-SOR, and pEG100:CTP-SOR-EGFP, respectively). In each case, the vector includes Bar as a selection marker, 35S as the promoter, attR sites, the 3′ sequences of the octapine synthase gene (OCS).

### Example 5

### Camelina sativa Transformation with Chloroplast Targeted SOR Sequences

Agrobacterium-mediated transformation was used to introduce the P. furiosus SOR into the chloroplasts, mitochondria, peroxisomes, and/or cell walls of C. sativa plants.

TABLE 2
Constructs used in transformation of Camelina.

| Construct | NOTES | Entry vector | Destination vector |
|---|---|---|---|
| 35S: GFP- | GFP on C | pCR8/GW/TOPO | pEG103→35S- |
| SOR | terminus | | GW-GFP-OCS-3′ |
| 35S:YFP- | YFP on N | pCR8/GW/TOPO | pEG104→35S- |
| SOR | terminus | | YFP-GW-OCS-3′ |
| 35S:eGFP- | GFP on N | pENTR/D/TOPO | pK7WGF2→35S- |
| SOR | terminus | | eGFP-GW-OCS-3′ |
| 35S: CTP- | No tags | pCR8/GW/TOPO | pEG100→35S-GW- |
| SOR | | | OCS-3′ |
| 35S: SOR | No tags | pENTR:SOR | pEG100→35S-GW- |
| | | | OCS-3′ |
| 35S: CTP- | CTP is in the | pCR8/GW/TOPO | pEG100→35S-GW- |
| egfp-SOR | N terminal | | OCS-3′ |
| | side for | | |
| | targeting to | | |
| | chloroplasts | | |

### Protocol for Transforming Camelina

Luria Broth (LB) medium for growing Agrobacterium

### Infiltration Medium:

1/2X MS salts

5% (w/v)Sucrose

0.044 uM BAP

0.05% Silwet L-77

### Procedure:

(1) Two days prior to transformation, a pre-culture of Agrobacterium carrying the appropriate binary vector is prepared by inoculating the Agrobacterium onto 3 ml LB medium including suitable antibiotics and incubating the culture at 28° C.
(2) One day prior to transformation a larger volume of (150 ml-300 ml) LB medium is inoculated with at least 1 ml of the preculture and incubated at 28° C. for about 16-24 hrs.
(3) Water plants prior to transformation.
(4) On the day of transformation of the plant, Agrobacterium cells are pelleted by centrifugation at 6000 rpm for 10 min at room temperature (e.g., about 19° C. to about 24° C.).
(5) The pellet is resuspended in 300-600 ml of infiltration medium (note: the infiltration medium is about double the volume used in the agro culture (about 150-300 ml)).
(6) The suspension solution is transferred to an open container that can hold the volume of infiltration medium prepared (300-600 ml) in which plants can be dipped and which fits into a desiccator.
(7) Place the container from (6) into a desiccator, invert a plant and dip the inflorescence shoots into the infiltration medium.
(8) Connect the desiccator to a vacuum pump and evacuate for 5 min at 16-85 kPa.
(9) Release the vacuum slowly.
(10) After releasing vacuum, remove the plants and orient them into an upright position or on their sides in a plastic nursery flat, and place a cover over them for the next 24 hours.
(11) The next day, the cover is removed, the plants rinsed with water and returned to their normal growing conditions (e.g., of about 22° C./18° C. (day/night) with daily watering under about 250-400 μE white light).
(12) A week later the plants were transformed again, repeating steps 1-11.
(13) The plants were watered on alternate days beginning after transformation for about 2-3 weeks and then twice a week for about another 2 weeks after which they were watered about once a week for about another 2-3 weeks for drying.

### Example 6

### Camelina sativa Transformation with Sequences Targeted to Mitochondria, Cell Wall, Cytosolic Membrane, and/or Peroxisome

### (1) Exemplary Targeting Sequences for Mitochondrial Targeting:

(SEQ ID NO: 26)
MLSLRQSIRFFKPATRTLCSSRYLLMISETIRSGDWKGEKHVPVIEYEREGELVKVK
VQVGKEIPHPNTTEHHIRYIELYFLPEGENFVYQVGRVEFTAHGESVNGPNTSDVYTE
PIAYFVLKTKKKGKLYALSYCNIHGLWENEVTLE
(SEQ ID NO: 27)
atgctgagcctgcgccagagcattcgcttttttaaaccggcgacccgcaccctgtgcagcagccgctatctgctg
atgattagcgaaaccattcgcagcggcgattggaaaggcgaaaaacatgtgccggtgattgaatatgaacgcgaa
ggcgaactggtgaaagtgaaagtgcaggtgggcaaagaaattccgcatccgaacaccaccgaacatcatattcgc
tatattgaactgtattttctgccggaaggcgaaaactttgtgtatcaggtgggccgcgtggaatttaccgcgcat
ggcgaaagcgtgaacggcccgaacaccagcgatgtgtataccgaaccgattgcgtattttgtgctgaaaaccaaa
aaaaaaggcaaactgtatgcgctgagctattgcaacattcatggcctgtgggaaaacgaagtgaccctggaa

### (2) Exemplary Targeting Sequences for Cell Wall Targeting

(SEQ ID NO: 28)
MRILPKSGGGALCLLFVFALCSVAHSMISETIRSGDWKGEKHVPVIEYEREGELVK
VKVQVGKEIPHPNTTEHHIRYIELYFLPEGENFVYQVGRVEFTAHGESVNGPNTSDV
YTEPIAYFVLKTKKKGKLYALSYCNIHGLWENEVTLE
(SEQ ID NO: 29)
atgcgcattctgccgaaaagaggcggcggcgcgctgtgcctgctgtttgtgtttgcgctgtgcagcgtggcgcat
agcatgattagcgaaaccattcgcagcggcgattggaaaggcgaaaaacatgtgccggtgattgaatatgaacgc
gaaggcgaactggtgaaagtgaaagtgcaggtgggcaaagaaattccgcatccgaacaccaccgaacatcatatt
cgctatattgaactgtattttctgccggaaggcgaaaactttgtgtatcaggtgggccgcgtggaatttaccgcg
catggcgaaagcgtgaacggcccgaacaccagcgatgtgtataccgaaccgattgcgtattttgtgctgaaaacc
aaaaaaaaaggcaaactgtatgcgctgagctattgcaacattcatggcctgtgggaaaacgaagtgaccctggaa

### (3) Exemplary Targeting Sequences for Peroxisomal Targeting.

(A) At the N terminus:
(SEQ ID NO: 30)
MRLSIHAEHLMISETIRSGDWKGEKHVPVIEYEREGELVKVKVQVGKEIPHPNTTEH
HIRYIELYFLPEGENFVYQVGRVEFTAHGESVNGPNTSDVYTEPIAYFVLKTKKKGKL
YALSYCNIHGLWENEVTLE
(SEQ ID NO: 31)
atgcgcctgagcattcatgcggaacatctgatgattagcgaaaccattcgcagcggcgattggaaaggcgaaaaa
catgtgccggtgattgaatatgaacgcgaaggcgaactggtgaaagtgaaagtgcaggtgggcaaagaaattccg
catccgaacaccaccgaacatcatattcgctatattgaactgtattttctgccggaaggcgaaaactttgtgtat
caggtgggccgcgtggaatttaccgcgcatggcgaaagcgtgaacggcccgaacaccagcgatgtgtataccgaa
ccgattgcgtattttgtgctgaaaaccaaaaaaaaaggcaaactgtatgcgctgagctattgcaacattcatggc
ctgtgggaaaacgaagtgaccctggaa
(B) At the C terminus:
(SEQ ID NO: 32)
MISETIRSGDWKGEKHVPVIEYEREGELVKVKVQVGKEIPHPNTTEHHIRYIELYFLPE
GENFVYQVGRVEFTAHGESVNGPNTSDVYTEPIAYFVLKTKKKGKLYALSYCNIHGL
WENEVTLESKL
(SEQ ID NO: 33)
atgattagcgaaaccattcgcagcggcgattggaaaggcgaaaaacatgtgccggtgattgaatatgaacgcgaa
ggcgaactggtgaaagtgaaagtgcaggtgggcaaagaaattccgcatccgaacaccaccgaacatcatattcgc
tatattgaactgtattttctgccggaaggcgaaaactttgtgtatcaggtgggccgcgtggaatttaccgcgcat
ggcgaaagcgtgaacggcccgaacaccagcgatgtgtataccgaaccgattgcgtattttgtgctgaaaaccaaa
aaaaaaggcaaactgtatgcgctgagctattgcaacattcatggcctgtgggaaaacgaagtgaccctggaaagc
aaactg

### (4) Exemplary Targeting Sequences for Targeting to the Cytosolic Membrane

CVVQ (SEQ ID NO:34)

(SEQ ID NO: 35)
tgtgtcgtgcag
SOR plus the targeting target peptide sequence:
(SEQ ID NO: 36)
MISETIRSGDWKGEKHVPVIEYEREGELVKVKVQVGKEIPHPNTTEHHIRYIELYFLPE
GENFVYQVGRVEFTAHGESVNGPNTSDVYTEPIAYFVLKTKKKGKLYALSDCNIHGL
WENEVTLECVVQ
SOR plus the targeting nucleotide sequence:
(SEQ ID NO: 37)
atgattagtgaaaccataagaagtggggactggaaaggagaaaagcacgtccccgttatagagtatgaaagagaa
ggggagcttgttaaagttaaggtgcaggttggtaaagaaatcccgcatccaaacaccactgagcaccacatcaga
tacatagagctttatttcttaccagaaggtgagaactttgtttaccaggttggaagagttgagtttacagctcac
ggagagtctgtaaacggcccaaacacgagtgatgtgtacacagaacccatagcttactttgtgctcaagactaag
aagaagggcaagctctatgctcttagcgactgtaacatccacggcctttgggaaaacgaagtcactttagagtgtg
tcgtgcag

The general motif for prenylation or farnesylation is C-terminal CaaX box motif on target proteins. C=cysteine; a=aliphatic amino acid; x=any amino acid. For plants, this motif most often is CVVQ (SEQ ID NO:34). This motif is required for specific posttranslational modifications (i.e. prenylation, farnesylation) that target the protein for association with the cytosolic side of the plasma membrane (the “inner” cytosolic side of the cell) or the cytosolic side of the nuclear membrane.

### (5) Exemplary Construct for Targeting to the Mitochondria and Chloroplast.

(SEQ ID NO: 38)
MLRTVSCLASRSSSSLFFRFFRQFPRSYMSLTSSTAALRVPSRNLRRISSPSVAGRRL
LLRRGLRIPSAAVRSVNGQFSRLSVRAMISETIRSGDWKGEKHYPVIEYEREGELVKV
KVQVGKEIPHPNTTEHHIRYIELYFLPEGENFVYQVGRVEFTAHGESVNGPNTSDVYT
EPIAYFVLKTKKKGKLYALSYCNIHGLWENEVTLE
(SEQ ID NO: 39)
atgctgcgcaccgtgagctgcctggcgagccgcagcagcagcagcctgttttttcgcttttttcgccagtttccg
cgcagctatatgagcctgaccagcagcaccgcggcgctgcgcgtgccgagccgcaacctgcgccgcattagcagc
ccgagcgtggcgggccgccgcctgctgctgcgccgcggcctgcgcattccgagcgcggcggtgcgcagcgtgaac
ggccagtttagccgcctgagcgtgcgcgcgatgattagcgaaaccattcgcagcggcgattggaaaggcgaaaaa
catgtgccggtgattgaatatgaacgcgaaggcgaactggtgaaagtgaaagtgcaggtgggcaaagaaattccg
catccgaacaccaccgaacatcatattcgctatattgaactgtattttctgccggaaggcgaaaactttgtgtat
caggtgggccgcgtggaatttaccgcgcatggcgaaagcgtgaacggcccgaacaccagcgatgtgtataccgaa
ccgattgcgtattttgtgctgaaaaccaaaaaaaaaggcaaactgtatgcgctgagctattgcaacattcatggc
ctgtgggaaaacgaagtgaccctggaa

### Example 7

### Analysis of Transformed C. saliva Plants

### (1) Verification of Expression in the Various Plant Organelles

RT-PCR and pRT-PCR Methods

RNA is isolated using the RNeasy kit (Qiagen), with an additional DNase I treatment to remove contaminating genomic DNA. Reverse transcription (RT) was carried out to generate cDNA using Omniscript reverse transcriptase enzyme (Qiagen). GFP-fused-SOR transcripts can be detected by PCR as described by Im et al., (2005) using internal GFP forward and gene specific primers (SOR reverse and actin specific primers), APX specific primers described in (Panchuk et al. Plant Physiol 129: 838-853 (2002) and Zat12 specific primers (forward; 5′ AACACAAACCACAAGAGGATCA 3′, SEQ ID NO:56, and reverse; 5′ CGTCAACGTTTTCTTGTCCA 3′, SEQ ID NO:57). Quantitative RT-PCR was carried out using Full Velocity SYBR-Green® QPCR Master Mix (Stratagene) on a MX3000P thermocycler (Stratagene). Gene specific primers for select genes were designed with the help of AtRTPrimer, a database for generating specific RT-PCR primer pairs (Han and Kim, BMC Bioinformatics 7:179 (2006)). Relative gene expression data were generated using the 2^{-ΔΔCt} method (Livak and Schmittgen, Methods 25:402-408 (2001)) using the wild-type zero time point as the reference. PCR conditions were 1 cycle of 95° C. for 10 min, 95° C. for 15 s, and 60° C. for 30 s to see the dissociation curve, 40 cycles of 95° C. for 1 minute for DNA denaturation, and 55° C. for 30 s for DNA annealing and extension.

### Immunoblotting (Western Analysis for SOR Detection)

Total protein extract is obtained from liquid N₂ frozen plants or seedlings grown as described by Weigel and Glazebrook, Arabidopsis: A Laboratory Manual. Cold Spring Harbor Laboratory Press, Cold Spring Harbor, N.Y. (2002)). Protein concentration is quantified as described by Bradford (Anal Biochem 72: 248-254, (1976)). Protein is separated by 10% (w/v) SDS-PAGE and detected with rabbit antibodies raised against P. furiosus SOR (at 1:2,000 dilution) or antibodies raised against HSP70, BiP, and CRT (at 1:1,000 dilution). Immunoreactivity is visualized with either horseradish peroxidase-conjugated anti-rabbit or anti-mouse antibodies (Pierce, Rockford, Ill.).

### SOR Activity Assay

Samples are ground with liquid nitrogen and lysed as described previously (Im et al., FEBS Lett 579: 5521-5526 (2005)). Samples are centrifuged at 27,000g at 4° C. for 30 min and resulting supernatants are passed through a 0.45 micron filter unit to remove cellular debris. Extracts are dialyzed overnight in 50 mM phosphate buffer. To reduce plant SOD background activity of dialyzed samples, samples are heat-treated (heat-treated at 80° C. for 15 min) and centrifuged at 21,000 g for 15 min. The heat treatments used are sufficient to inactivate some endogenous plant SOD activity, allowing for greater discrimination between SOD and SOR activity in the transgenic plants. To avoid leaf pigments and reduce loss of activity resulting from dialysis, roots are harvested from seedlings grown for 28 days or 42 days on agar plates in a growth chamber (8 h light/16 h dark).

The standard SOD/SOR assay is performed as described in Im et al. (FEBS Lett 579: 5521-5526 (2005)). One unit of SOD/SOR activity is defined as the amount of enzyme that inhibits the rate of reduction of cytochrome c by 50% (McCord and Fridovich, J Biol Chem 244: 6049-6055 (1969)).

### (2) Reduction in ROS

### H₂O₂ Measurements (FOX Assay)

A ferrous ammonium sulfate/xylenol orange (FOX) method is used to quantify H₂O₂ in plant extracts (Wolff, Methods Enzymol 233: 182-189, 1994)). The original FOX method is modified by addition of an acidification step where 1 ml of 25 mM H₂SO₄ was added to each sample to allow for precipitation of interfering substances (sugars, starches, polysaccharides) for 15 min on ice, and centrifuged at 9,700 g, for 15 min, at 4° C. The cell free extract is collected and passed through a 0.45 μm-filter unit. 100 μl is added to 1 ml of the FOX reagent, mixed, and incubated at room temperature for 20 min. The concentration of H₂O₂ in the reagent is calibrated using absorbance at 240 nm and an extinction coefficient of 43.6 M⁻¹ cm⁻¹. The concentration of H₂O₂ is measured in nmoles H₂O₂ per gram of fresh wt cells.

### Ascorbate Peroxidase (APX) Activity Assay

APX activity is determined as described previously (Nakano and Asada, Plant Cell Physiol 22:867-880, 1981). Fifty μg of the extract is used in a 3 ml APX assay and the reaction proceeds for 2 minutes. APX activity is expressed as μmol of ascorbate oxidized (mg protein)⁻¹ min⁻¹. Additional confirmation of APX activity can be done by an in-gel assay as described by Panchuk et al. (Plant Physiol 129: 838-853 (2002)).

### (3) Protection of the Photosynthetic Apparatus and its Surrounding Membrane Lipids

To quantify the protection of the photosystems, leaf fluorescence and CO2 fixation rates of fully expanded leaves is measured using a LiCOR system. The maximal photochemical efficiency of the PSII is calculated using the ratio Fᵥ/Fₘ, where Fᵥ=Fₘ−Fₒ (Genty et al., Biochimica et Biophysica Acta (BBA)—General Subjects 990: 87-92 (1989)). This is calculated from initial (Fₒ) and maximum fluorescence (Fₘ) as measured in vivo on the last fully expanded leaf pre-acclimatized to the dark for approximately 40 min. Fₘ can be estimated by applying a light saturating flash with an intensity of ca. 8,000 μmol photons m⁻² s⁻¹.

### (4) Reduction in Photorespiration

Reduction in photorespiration is determined by CO₂ fixation rates as described above using a LICOR system, Plants are exposed to atmospheric CO₂:O₂ mixtures (400 ppm CO₂/21% O₂) or at saturating CO₂ concentrations (4000 ppm/21% O₂) and their biomass, photosynthetic CO₂ fixation rates, chlorophyll fluorescence and chlorophyll content are quantified. Higher CO₂ fixation rates in the transgenic plants under limiting CO₂ compared to wild type and control plants indicate reduced photorespiratory activity.

### (5) Increased Tolerance to Abiotic Stress

### Thermotolerance Assays

To test seed basal thermotolerance, stratified seeds are treated at 45° C. for 5 h and germination was evaluated 2 days (d) later following the protocol of Larkindale et al. Plant Physiol 138: 882-897 (2005). The hypocotyl elongation assay was carried out as described by Hong and Vierling, (Proc Natl Acad Sci USA 97: 4392-4397 (2000)). Growth after the heat treatment was measured and compared with that of seedlings receiving no heat treatment. For tests of vegetative-stage plants, 10 day-old grown seedlings were used as described by Hong and Vierling (Proc Natl Acad Sci USA 97: 4392-4397 (2000)). Heat-treated plates were returned to the 22° C. incubator and all plates were left at 22° C. for 7 d. The number of seedlings that survived were counted after 7 d.

Mature, flowering plants grown at 22° C. are exposed for 0 days, 2 days, 4 days, 6 days and 10 days to 35° C. Survival rate, seed set, flower number, chlorophyll content and total final seed number, seed weight and seed germination rate is analyzed per plant.

### Quantification of Chlorophyll for Plants Exposed to Heat Challenge

Etiolated seedlings were grown for 2.5 days in the dark at 22° C.; exposed to 48° C. for 30 min in the dark, and transferred to continuous light for 24 hrs. Seedlings were ground with liquid nitrogen and extracted with 80% (v/v) acetone by shaking until the leaves became bleached. The chlorophyll content in the acetone extract was quantified spectrophotometrically based on absorbance at 663 nm as described by (Burke et al. Plant Physiol. 123:575-588 (2000)).

### SOR Protection Against Chemically Induced ROS

Seeds (25 seeds of each line) are sterilized and plated on a single plate of 0.8% MS medium containing different concentrations of paraquat (0, 0.25, 0.5 and 1 μM). Plant survival (number of green seedlings) is calculated for each line after 14 d under continuous light. Results are reported as percent of each control (100%) and show mean±SD from 3 independent experiments.

### (6) Reduction in Lignin Polymerization

### Histochemical Staining

In order to examine the lignified cell walls in stems, the transgenic and WT plants are grown under the same conditions for 2 months. The second internodes of stems (from ground level) are excised, the bark removed, and the internodes hand-cut into 20-30 μm thick slices, and subjected to histochemical analysis. Wiesner staining is performed by incubating sections in 1% phloroglucinol (w/v) in 6 mol 1⁻¹HCl for 5 min, and the sections observed under a dissecting microscope (Pomar et al., Protoplasma 220:17-28 (2002); Weng et al., The Plant Cell 22, 1033-1045 (2010). For Mäule staining, hand-cut stem sections are soaked in 1% KMnO₄ for 5 min, then rinsed with water, destained in 30% HCl, washed with water, mounted in concentrated NH₄OH, and examined under a dissecting microscope (Atanassova et al., The Plant Journal 8, 465-477 (1995); Weng et al., The Plant Cell 22, 1033-1045 (2010)).

### Assay of Klason Lignin Content

The second internodes of stems (from ground level) of transgenic and WT plants grown under the same conditions for approximately 2 months, are excised, the bark removed, and the internodes then cut into thin sections and put into an 80° C. oven. The dried stem materials are ground into a fine powder, extracted four times in methanol and dried. Then 200 mg of the extract is mixed with 5 ml of 72% (w/w) sulfuric acid at 30° C. and hydrolyzed for 1 h. The hydrolysate was diluted to 4% sulfur by the addition of water and then cooked for 1 h in boiling water. The solid residue is filtered through a glass filter. Finally, the sample is washed, dried at 80° C. overnight and then weighed. The lignin content is measured and expressed as a percentage of the original weight of cell wall residue (Dence C. 1992. Lignin determination. In: Lin S, ed., Methods in lignin chemistry. Berlin: Springer-Verlag, 33-61).

### (7) Increased Accessibility to Cell Wall Cellulose by an Enzyme

### Cellulose Accessibility

The cellulose accessibility of biomass and the pure cellulose samples is determined using fluorescence-labeled, purified Trichoderma reesei Cel7A. Triplicate samples (250 mL final volume) containing 1.0 mM T. reesei Cel7A with a substrate concentration equivalent to 1.0 mg mL⁻¹ final cellulose concentration in 5 mM sodium acetate pH 5.0 buffer are prepared for each reaction time assayed throughout a 120 h time course. Reactions are conducted at 38° C., rotating end-over-end and assayed at 1, 4, 24, 48, and 120 h. Each reaction is initiated by the addition of enzyme and terminated by filtration in a 96-well vacuum filter manifold (Innovative Microplate, Chicopee, Mass.) equipped with a 1.0 mm glass fiber filter. The reaction supernatant is assayed for reducing sugars using the BCA method (Doner and Irwin, Anal Biochem 202(1):50-531992) against a cellobiose standard curve. The solid fraction retained in the filter was assayed for bound T. reesei Cel7A concentration.

### Bound Cellulase Enzyme Quantitation

The concentration of bound enzyme on the solids fraction from the accessibility experiments is assayed by fluorometry with adjustments for biomass autofluorescence. Following filtration of the reaction samples, the retained solids (containing pure cellulose samples (PCS) bound T. reesei Cel7A) are resuspended with 250 mL of distilled water. For each sample, 150 mL of the resuspended solids are transferred to a microtiter plate and read in a FLUOstar optima plate reader (BMG Labtechnologies, Durham, N.C.) at excitation and emission wavelengths of 584 and 612 nm, respectively. The emission intensities from the samples are converted to concentrations of T. reesei Cel7A using regression parameters from a standard curve of calibration standards that are measured concurrently. To negate the autofluorescence of each of the PCS, a separate calibration is made for each PCS sample digested with Cel7A. The calibration curves contain six levels of standard additions (0-1 mM T. reesei Cel7A) with the same concentration of PCS as used in each of the accessibility experiments. To negate the effects of plate-to-plate or day-to-day variations in the fluorescence measurements, a fresh set of calibration standards (in triplicate, with the appropriate PCS sample) is included with each microtiter plate containing unknown samples from the reactions.

The effect of digestion on the correction of autofluorescence in the calibration standards is examined as follows. Fifteen replicates of a PCS sample are digested to 67^{±}9% by unlabeled T. reesei Cel7A in 5-days, using the conditions described above for the cellulase accessibility experiments. The reactions are terminated by filtration and the solids fractions re-suspended in 125 mL of distilled water. The re-suspended solids are transferred to a microtiter plate, with 75 mL from each replicate pipette into each well. Standard additions of fluorescence—labeled T. reesei Cel7A including five levels ranging from 0.12 to 2 mM are prepared. Each amount is pipetted in triplicate (75 mL per replicate) to the wells containing digested PCS. Calibration standards with the same final T. reesei concentrations are then prepared in the same microtiter plate, using undigested PCS. The plate is read in the fluorometer as described earlier. The concentrations of T. reesei Cel7A with the digested PCS are determined using regression parameters from the standard curve developed using the undigested PCS. These values are compared to the expected values to determine the effect of extensive digestion on the quantitation method.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

1. A stably transformed plant, plant cell, plant part, yeast cell or bacterial cell comprising a heterologous polynucleotide encoding a superoxide reductase (SOR) from an archaeon species, wherein the superoxide reductase is localized in the cytosolic membrane, the chloroplast, the peroxisome, the cell wall, mitochondria, periplasm and/or as a membrane associated protein of the transformed plant, plant cell, plant part, yeast cell or bacterial cell.

2. The stably transformed plant, plant cell, plant part, yeast cell or bacterial cell of claim 1, wherein the archaeon species is a species from the genus Pyrococcus, a species from the genus Thermococcus, or a species from the genus Archaeoglobus.

3. The stably transformed plant, plant cell, plant part, yeast cell or bacterial cell of claim 1, wherein the archaeon species is Pyrococcus furiosus.

4. The stably transformed plant, plant cell, plant part, yeast cell or bacterial cell of claim 1, wherein the heterologous polynucleotide encodes a nucleotide sequence having substantial identity to the nucleotide sequence of SEQ ID NO:1 or SEQ ID NO:2.

5. The stably transformed plant, plant cell, plant part, yeast cell or bacterial cell of claim 1, wherein the heterologous polynucleotide is operably associated with a targeting sequence.

6. The stably transformed plant, plant cell, plant part, yeast cell or bacterial cell of claim 1, wherein the heterologous polynucleotide encodes an amino acid sequence having substantial identity to an amino acid sequence of SEQ ID NO:3 or SEQ ID NO:4.

7. The stably transformed plant, plant cell, plant part, yeast cell or bacterial cell of claim 1, wherein the plant, plant cell, plant part, yeast cell or bacterial cell further comprises a heterologous polynucleotide encoding a rubrerythrin reductase from an archaeon species, wherein the rubrerythrin reductase is co-localized with the SOR in said transformed plant, plant cell, plant part, yeast cell or bacterial cell.

8. The stably transformed plant, plant cell, plant part, yeast cell or bacterial cell of claim 7, wherein the rubrerythrin reductase is encoded by a nucleotide sequence that encodes the amino acid sequence of SEQ ID NO:51.

9. A seed of the stably transformed plant of claim 1, wherein the seed comprises in its genome a heterologous polynucleotide encoding superoxide reductase from an archaeon species.

10. A seed of the stably transformed plant of claim 7, wherein the seed comprises in its genome a heterologous polynucleotide encoding superoxide reductase from an archaeon species and a heterologous polynucleotide encoding a rubrerythrin reductase from an archaeon species.

11. A product produced from the stably transformed plant, plant cell, plant part, yeast cell or bacterial cell of claim 1.

12. A product produced from the seed of claim 10.

13. The product of claim 11, wherein the product is biofuel, food, drink, animal feed, fiber, and/or pharmaceuticals.

14. The product of claim 12, wherein the product is biofuel, food, drink, animal feed, fiber, and/or pharmaceuticals.

15. The stably transformed plant, plant part and/or plant cell of claim 1, wherein the superoxide reductase is expressed and localized to
(a) the cytosolic membrane, the chloroplast, the peroxisome, the cell wall, mitochondria and/or as a membrane associated protein of the stably transformed plant, plant part and/or plant cell and the stably transformed plant, plant part and/or plant cell has reduced reactive oxygen species;
(b) the chloroplasts of the stably transformed plant, plant part, and/or plant cell and the stably transformed plant, plant part and/or plant cell has a protected photosynthetic reaction center;
(c) the chloroplast, the peroxisome and/or the mitochondria of the stably transformed plant, plant cell, plant part and/or plant cell and the stably transformed plant, plant part and/or plant cell has reduced photorespiration and/or increased photosynthetic efficiency;
(d) the chloroplast, the cell wall, mitochondria and/or as a membrane associated protein of the stably transformed plant, plant part and/or plant cell and the stably transformed plant, plant part and/or plant cell has increased tolerance to heat, high light, drought, ozone, heavy metals, pesticides, herbicides, toxins, and/or anoxia.
(e) the cell wall of the stably transformed plant, plant part and/or plant cell and the stably transformed plant, plant part and/or plant cell has reduced lignin polymerization;
(f) the cell wall of the stably transformed plant, plant part and/or plant cell and the accessibility of the cell wall cellulose of the stably transformed plant, plant part and/or plant cell to at least one enzyme is increased; and/or
(g) the chloroplast, mitochondria, peroxisome, cytosolic membrane, and/or mitochondria of the stably transformed plant, plant part and/or plant cell and the stably transformed plant, plant part, and/or plant cell has delayed senescence.

16. An expression cassette comprising an isolated nucleic acid having:
(a) a nucleotide sequence of SEQ ID NO:2;
(b) a nucleotide sequence that encodes a polypeptide comprising an amino acid sequence of SEQ ID NO: 4; and/or
(c) a nucleotide sequence that differs from the nucleotide sequences of (a) or (b) above due to the degeneracy of the genetic code.

17. A method of reducing reactive oxygen species in a plant, plant cell, plant part, yeast cell or bacterial cell, comprising:
introducing into the plant, plant cell, plant part, yeast cell or bacterial cell a heterologous polynucleotide encoding a superoxide reductase from an archaeon species to produce the stably transformed plant, plant cell, plant part, yeast cell or bacterial cell of claim 1, wherein the superoxide reductase is expressed and localized to the cytosolic membrane, the chloroplast, the peroxisome, the cell wall, mitochondria, periplasm and/or as a membrane associated protein of the stably transformed plant, plant cell, plant part, yeast cell or bacterial cell, thereby reducing reactive oxygen species in the stably transformed plant, plant cell, plant part, yeast cell or bacterial cell.

18. A method of protecting a photosynthetic apparatus and surrounding membranes of a plant, plant cell, plant part, or bacterial cell, comprising:
introducing into the plant, plant cell, plant part, or bacterial cell a heterologous polynucleotide encoding a superoxide reductase from an archaeon species to produce the stably transformed plant, plant cell, plant part, or bacterial cell of claim 1, wherein the bacterial cell is a cyanobacteria cell and the superoxide reductase is expressed and localized to the chloroplasts of the stably transformed plant, plant cell, plant part, or the cytosol of cyanobacteria cell, thereby protecting the photosynthetic reaction center of the stably transformed plant, plant cell, plant part, or cyanobacteria cell.

19. A method of reducing photorespiration in a plant, plant cell, plant part, or cyanobacteria cell, comprising:
introducing into the plant, plant cell, plant part, or bacterial cell a heterologous polynucleotide encoding a superoxide reductase from an archaeon species to produce the stably transformed plant, plant cell, plant part, or bacterial cell of claim 1, wherein the bacterial cell is a cyanobacteria cell and the superoxide reductase is expressed and localized to the chloroplast, the peroxisome, and/or the mitochondria of the stably transformed plant, plant cell, plant part, or cyanobacteria cell, thereby reducing photorespiration in the stably transformed plant, plant cell, plant part, or cyanobacteria cell.

20. A method of increasing photosynthetic efficiency in a plant, plant cell, plant part, or bacterial cell, comprising:
introducing into the plant, plant cell, plant part, or bacterial cell a heterologous polynucleotide encoding a superoxide reductase from an archaeon species to produce a stably transformed plant, plant cell, plant part, or bacterial cell of claim 1, wherein the bacterial cell is a cyanobacteria cell and the superoxide reductase is expressed and localized to the chloroplasts of the stably transformed plant, plant cell, plant part, or cyanobacteria cell, thereby increasing photosynthetic efficiency in the stably transformed plant, plant cell, plant part, or cyanobacteria cell.

21. A method of increasing tolerance to heat, high light, drought, ozone, heavy metals, pesticides, herbicides, toxins, and/or anoxia in a plant, plant cell, plant part, yeast cell or bacterial cell, comprising:
introducing into the plant, plant cell, plant part, yeast cell or bacterial cell a heterologous polynucleotide encoding a superoxide reductase from an archaeon species to produce the stably transformed plant, plant cell, plant part, yeast cell or bacterial cell of claim 1, wherein the superoxide reductase is expressed and localized to the chloroplast, the cell wall mitochondria, periplasm and/or as a membrane associated protein, of the stably transformed plant, plant cell, plant part, yeast cell or bacterial cell, thereby increasing tolerance to heat, high light, drought, ozone, heavy metals, pesticides, herbicides, toxins, and/or anoxia in the stably transformed plant, plant cell, plant part, yeast cell or bacterial cell.

22. A method of reducing lignin polymerization in a plant, plant part and/or plant cell, comprising:
introducing into the plant, plant part and/or plant cell a heterologous polynucleotide encoding a superoxide reductase from an archaeon species to produce the stably transformed plant, plant part and/or plant cell of claim 1, wherein the superoxide reductase is expressed and localized to the cell wall of the stably transformed plant, plant part and/or plant cell, thereby reducing lignin polymerization in the stably transformed plant and/or plant part.

23. A method of increasing accessibility of cell wall cellulose in a plant, plant part and/or plant cell part to at least one enzyme, comprising:
introducing into the plant, plant part and/or plant cell a heterologous polynucleotide encoding a superoxide reductase from an archaeon species to produce the stably transformed plant, plant part and/or plant cell of claim 1, wherein the superoxide reductase is expressed and localized to the cell wall of the stably transformed plant, plant part and/or plant cell, thereby increasing accessibility of the cell wall cellulose to at least one enzyme in the stably transformed plant, plant part and/or plant cell.

24. A method of delaying senescence in a plant, plant part, plant cell, bacterium and/or yeast, comprising:
introducing into the plant, plant part, plant cell, bacterium and/or yeast a heterologous polynucleotide encoding a superoxide reductase from an archaeon species to produce the stably transformed plant, plant part, plant cell, bacterium and/or yeast of claim 1, wherein the superoxide reductase is expressed and localized to the chloroplast, mitochondria, peroxisome, cytosolic membrane, and/or mitochondria of the stably transformed plant, plant part, plant cell, bacterium and/or yeast, thereby delaying the senescence of the stably transformed plant, plant part, plant cell, bacterium and/or yeast.


SEQUENCE LISTING (57 sequences)

SEQ ID NO: 1 - DNA, 375 bp, Pyrococcus furiosus
atgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagctactgtaacatccacggcctttgggaaaacgaagtcactttagagtga

SEQ ID NO: 2 - DNA, 375 bp, Pyrococcus furiosus
atgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagcgactgtaacatccacggcctttgggaaaacgaagtcactttagagtga

SEQ ID NO: 3 - protein, 124 aa, Pyrococcus furiosus
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 4 - protein, 124 aa, Pyrococcus furiosus
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSDCNIHGLWENEVTLE

SEQ ID NO: 5 - protein, 57 aa, Nicotiana tabacum
MASSVLSSAAVATRSNVAQANMVAPFTGLKSAASFPVSRKQNLDITSIASNGGRVQC

SEQ ID NO: 6 - DNA, 171 bp, Nicotiana tabacum
atggcgagcagcgtgctgagcagcgcggcggtggcgacccgcagcaacgtggcgcaggcgaacatggtggcgccgtttaccggcctgaaaagcgcggcgagctttccggtgagccgcaaacagaacctggatattaccagcattgcgagcaacggcggccgcgtgcagtgc

SEQ ID NO: 7 - protein, 25 aa, Saccharomyces cerevisiae
MLSLRQSIRFFKPATRTLCSSRYLL

SEQ ID NO: 8 - DNA, 75 bp, Saccharomyces cerevisiae
atgctgagcctgcgccagagcattcgcttttttaaaccggcgacccgcaccctgtgcagcagccgctatctgctg

SEQ ID NO: 9 - protein, 78 aa, Arabidopsis thaliana
MYLTASSSASSSIIRAASSRSSSLFSFRSVLSPSVSSTSPSSLLARRSFGTISPAFRRWSHSFHSKPSPFRFTSQIRA

SEQ ID NO: 10 - DNA, 234 bp, Arabidopsis thaliana
atgtatctgaccgcgagcagcagcgcgagcagcagcattattcgcgcggcgagcagccgcagcagcagcctgtttagctttcgcagcgtgctgagcccgagcgtgagcagcaccagcccgagcagcctgctggcgcgccgcagctttggcaccattagcccggcgtttcgccgctggagccatagctttcatagcaaaccgagcccgtttcgctttaccagccagattcgcgcg

SEQ ID NO: 11 - protein, 26 aa, Saccharomyces cerevisiae
MRILPKSGGGALCLLFVFALCSVAHS

SEQ ID NO: 12 - DNA, 78 bp, Saccharomyces cerevisiae
atgcgcattctgccgaaaagcggcggcggcgcgctgtgcctgctgtttgtgtttgcgctgtgcagcgtggcgcatagc

SEQ ID NO: 13 - protein, 9 aa, Artificial
  Consensus PTS-2 sequence
RLXXXXXHL

SEQ ID NO: 14 - protein, 10 aa, Artificial
  Peroxisome signal sequence
MRLSIHAEHL

SEQ ID NO: 15 - DNA, 30 bp, Artificial
  Peroxisome signal coding sequence
atgcgcctgagcattcatgcggaacatctg

SEQ ID NO: 16 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 17 - DNA, 255 bp, Arabidopsis thaliana
atgctgcgcaccgtgagctgcctggcgagccgcagcagcagcagcctgttttttcgcttttttcgccagtttccgcgcagctatatgagcctgaccagcagcaccgcggcgctgcgcgtgccgagccgcaacctgcgccgcattagcagcccgagcgtggcgggccgccgcctgctgctgcgccgcggcctgcgcattccgagcgcggcggtgcgcagcgtgaacggccagtttagccgcctgagcgtgcgcgcg

SEQ ID NO: 18 - protein, 181 aa, Artificial
  Chloroplast targeted SOR sequence
MASSVLSSAAVATRSNVAQANMVAPFTGLKSAASFPVSRKQNLDITSIASNGGRVQCMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 19 - DNA, 543 bp, Artificial
  Chloroplast targeted SOR coding sequence
atggcgagcagcgtgctgagcagcgcggcggtggcgacccgcagcaacgtggcgcaggcgaacatggtggcgccgtttaccggcctgaaaagcgcggcgagctttccggtgagccgcaaacagaacctggatattaccagcattgcgagcaacggcggccgcgtgcagtgcatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 20 - protein, 425 aa, Artificial
  Chloroplast targeted EGFP-SOR fusion protein sequence
MASSVLSSAAVATRSNVAQANMVAPFTGLKSAASFPVSRKQNLDITSIASNGGRVQCMQMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLSSMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 21 - DNA, 1269 bp, Artificial
  Chloroplast targeted EGFP-SOR fusion protein coding sequence
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagctactgtaacatccacggcctttgggaaaacgaagtcactttagagtga

SEQ ID NO: 22 - protein, 422 aa, Artificial
  Chloroplast targeted SOR-EGFP fusion protein sequence
MASSVLSSAAVATRSNVAQANMVAPFTGLKSAASFPVSRKQNLDITSIASNGGRVQCMQMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - DNA, 1269 bp, Artificial
  Chloroplast targeted SOR-EGFP fusion protein sequence
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcagatgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagctactgtaacatccacggcctttgggaaaacgaagtcactttagagtgaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 24 - protein, 419 aa, Artificial
  CHloroplast targeting YFP-SOR fusion protein sequence
MASSVLSSAAVATRSNVAQANMVAPFTGLKSAASFPVSRKQNLDITSIASNGGRVQCMGKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 25 - DNA, 1257 bp, Artificial
  Chloroplast targeting YFP-SOR coding sequence
atggcgagcagcgtgctgagcagcgcggcggtggcgacccgcagcaacgtggcgcaggcgaacatggtggcgccgtttaccggcctgaaaagcgcggcgagctttccggtgagccgcaaacagaacctggatattaccagcattgcgagcaacggcggccgcgtgcagtgcatgggcaaaggcgaagaactgtttaccggcgtggtgccgattctggtggaactggatggcgatgtgaacggccataaatttagcgtgagcggcgaaggcgaaggcgatgcgacctatggcaaactgaccctgaaatttatttgcaccaccggcaaactgccggtgccgtggccgaccctggtgaccacctttggctatggcctgcagtgctttgcgcgctatccggatcatatgaaacagcatgatttttttaaaagcgcgatgccggaaggctatgtgcaggaacgcaccattttttttaaagatgatggcaactataaaacccgcgcggaagtgaaatttgaaggcgataccctggtgaaccgcattgaactgaaaggcattgattttaaagaagatggcaacattctgggccataaactggaatataactataacagccataacgtgtatattatggcggataaacagaaaaacggcattaaagtgaactttaaaattcgccataacattgaagatggcagcgtgcagctggcggatcattatcagcagaacaccccgattggcgatggcccggtgctgctgccggataaccattatctgagctatcagagcgcgctgagcaaagatccgaacgaaaaacgcgatcatatggtgctgctggaatttgtgaccgcggcgggcattaccctgggcatggatgaactgtataaaatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 26 - protein, 149 aa, Artificial
  Mitochondrial targeting SOR protein sequence
MLSLRQSIRFFKPATRTLCSSRYLLMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 27 - DNA, 447 bp, Artificial
  Mitochondrial targeting SOR coding sequence
atgctgagcctgcgccagagcattcgcttttttaaaccggcgacccgcaccctgtgcagcagccgctatctgctgatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 28 - protein, 150 aa, Artificial
  Cell wall targeting SOR protein sequence
MRILPKSGGGALCLLFVFALCSVAHSMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 29 - DNA, 450 bp, Artificial
  Cell wall targeting SOR coding sequence
atgcgcattctgccgaaaagcggcggcggcgcgctgtgcctgctgtttgtgtttgcgctgtgcagcgtggcgcatagcatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 30 - protein, 134 aa, Artificial
  Peroxisomal targeting SOR protein sequence
MRLSIHAEHLMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 31 - DNA, 402 bp, Artificial
  Peroxisomal targeting SOR coding sequence
atgcgcctgagcattcatgcggaacatctgatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 32 - protein, 127 aa, Artificial
  Peroxisomal targeting SOR protein sequence
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLESKL

SEQ ID NO: 33 - DNA, 381 bp, Artificial
  Peroxisomal targeting SOR coding sequence
atgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaaagcaaactg

SEQ ID NO: 34 - protein, 4 aa, Artificial
  Cytosolic membrane targeting sequence
CVVQ

SEQ ID NO: 35 - DNA, 12 bp, Artificial
  Cytosolic membrane targeting coding sequence
tgtgtcgtgcag

SEQ ID NO: 36 - protein, 128 aa, Artificial
  Cytosolic membrane targeting SOR protein sequence
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSDCNIHGLWENEVTLECVVQ

SEQ ID NO: 37 - DNA, 384 bp, Artificial
  Cytosolic membrane SOR coding sequence
atgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagcgactgtaacatccacggcctttgggaaaacgaagtcactttagagtgtgtcgtgcag

SEQ ID NO: 38 - protein, 209 aa, Artificial
  Mitochondrial and chloroplast targeting SOR protein sequence
MLRTVSCLASRSSSSLFFRFFRQFPRSYMSLTSSTAALRVPSRNLRRISSPSVAGRRLLLRRGLRIPSAAVRSVNGQFSRLSVRAMISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 39 - DNA, 627 bp, Artificial
  Mitochondrial and chloroplast targeting SOR coding sequence
atgctgcgcaccgtgagctgcctggcgagccgcagcagcagcagcctgttttttcgcttttttcgccagtttccgcgcagctatatgagcctgaccagcagcaccgcggcgctgcgcgtgccgagccgcaacctgcgccgcattagcagcccgagcgtggcgggccgccgcctgctgctgcgccgcggcctgcgcattccgagcgcggcggtgcgcagcgtgaacggccagtttagccgcctgagcgtgcgcgcgatgattagcgaaaccattcgcagcggcgattggaaaggcgaaaaacatgtgccggtgattgaatatgaacgcgaaggcgaactggtgaaagtgaaagtgcaggtgggcaaagaaattccgcatccgaacaccaccgaacatcatattcgctatattgaactgtattttctgccggaaggcgaaaactttgtgtatcaggtgggccgcgtggaatttaccgcgcatggcgaaagcgtgaacggcccgaacaccagcgatgtgtataccgaaccgattgcgtattttgtgctgaaaaccaaaaaaaaaggcaaactgtatgcgctgagctattgcaacattcatggcctgtgggaaaacgaagtgaccctggaa

SEQ ID NO: 40 - protein, 19 aa, Saccharomyces cerevisiae
MLSARSAIKRPIVRGLATV

SEQ ID NO: 41 - protein, 35 aa, Chlamydomonas reinhardtii
MALVARPVLSARVAASRPRVAARKAVRVSAKYGEN

SEQ ID NO: 42 - protein, 29 aa, Chlamydomonas reinhardtii
MQALSSRVNIAAKPQRAQRLVVRAEEVKA

SEQ ID NO: 43 - protein, 35 aa, Chlamydomonas reinhardtii
MQTLASRPSLRASARVAPRRAPRVAVVTKAALDPQ

SEQ ID NO: 44 - protein, 31 aa, Chlamydomonas reinhardtii
MQALATRPSAIRPTKAARRSSVVVRADGFIG

SEQ ID NO: 45 - protein, 51 aa, Chlamydomonas reinhardtii
MAFALASRKALQVTCKATGKKTAAKAAAPKSSGVEFYGPNRAKWLGPYSEN

SEQ ID NO: 46 - protein, 50 aa, Chlamydomonas reinhardtii
MAAVIAKSSVSAAVARPARSSVRPMAALKPAVKAAPVAAPAQANQMMVWT

SEQ ID NO: 47 - protein, 40 aa, Chlamydomonas reinhardtii
MAAMLASKQGAFMGRSSFAPAPKGVASRGSLQVVAGLKEV

SEQ ID NO: 48 - protein, 21 aa, Escherichia coli
MKQSTIAKAKKPLLFTPVTKA

SEQ ID NO: 49 - protein, 9 aa, Artificial
  Exemplary peroxisomal targeting sequence
RLAVAVAHL

SEQ ID NO: 50 - DNA, 516 bp, Pyrococcus furiosus
atggtcgtgaaaagaacaatgactaaaaagttcttggaagaagcctttgcaggcgaaagcatggcccatatgaggtatttgatctttgccgagaaagctgaacaagaaggatttccaaacatagccaagctgttcagggcaatagcttacgcagagtttgttcacgctaaaaaccacttcatagctctaggaaaattaggcaaaactccagaaaacttacagatgggaatagagggagaaacgttcgaagttgaggaaatgtacccagtatacaacaaagccgcagaattccaaggagaaaaggaagcagttagaacaacccactatgctttagaggcggagaagatccacgctgaactctatagaaaggcaaaagagaaagctgagaaaggggaagacattgaaataaagaaagtttacatatgcccaatctgtggatacaccgctgttgatgaggctccagaatactgtccagtttgtggagctccaaaagaaaagttcgttgtctttgaatga

SEQ ID NO: 51 - protein, 171 aa, Pyrococcus furiosus
MVVKRTMTKKFLEEAFAGESMAHMRYLIFAEKAEQEGFPNIAKLFRAIAYAEFVHAKNHFIALGKLGKTPENLQMGIEGETFEVEEMYPVYNKAAEFQGEKEAVRTTHYALEAEKIHAELYRKAKEKAEKGEDIEIKKVYICPICGYTAVDEAPEYCPVCGAPKEKFVVFE

SEQ ID NO: 52 - DNA, 58 bp, Escherichia coli
atgaaacagagcaccattgcgaaagcgaaaaaaccgctgctgtttaccccggtgacca

SEQ ID NO: 53 - DNA, 24 bp, Artificial
  PCR primer
caccatgattagtgaaaccataag

SEQ ID NO: 54 - DNA, 20 bp, Artificial
  PCR primer
atgattagtgaaaccataag

SEQ ID NO: 55 - DNA, 24 bp, Artificial
  PCR primer
tcactctaaagtgacttcgttttc

SEQ ID NO: 56 - DNA, 22 bp, Artificial
  PCR primer
aacacaaaccacaagaggatca

SEQ ID NO: 57 - DNA, 20 bp, Artificial
  PCR primer
cgtcaacgttttcttgtcca